(19) United States
(12) Patent Application Publication
(10) Pub. No.: US 2025/0264482 A1
(43) Pub. Date: Aug. 21, 2025

(54) STEREOTYPIC BCR CLONOTYPES IN ALZHEIMER'S DISEASE PATIENTS AND USE THEREOF

(71) Applicant: Seoul National University R&DB Foundation, Seoul (KR)

(72) Inventors: Junho Chung, Seoul (KR); Hyunji Yang, Seoul (KR); Duck Kyun Yoo, Seoul (KR); Ji Won Han, Seongnam-si (KR); Ki Woong Kim, Seoul (KR); Jong Bin Bae, Seoul (KR); Sunghoon Kwon, Seoul (KR); Yonghee Lee, Seoul (KR); Namphil Kim, Hanam-si (KR)

(73) Assignee: Seoul National University R&DB Foundation, Seoul (KR)

(21) Appl. No.: 19/195,699

(22) Filed: Apr. 30, 2025

Related U.S. Application Data
(63) Continuation-in-part of application No. 18/827,742, filed on Sep. 7, 2024.

(30) Foreign Application Priority Data
Sep. 7, 2023 (KR) 10-2023-0118789

(51) Int. Cl. G01N 33/68; C07K 16/28

(52) U.S. Cl. CPC G01N 33/6896; C07K 16/2803; C07K 2317/622

(57) ABSTRACT

The present specification discloses a composition, a system, and a method for predicting or diagnosing Alzheimer's disease, comprising a detecting agent of a B cell receptor clonotype specific to an Alzheimer's disease patient, and the composition according to one aspect of the present invention has an excellent effect in predicting or diagnosing Alzheimer's disease by simply using peripheral blood mononuclear cells isolated from a subject, in addition to methods such as MRI and PET scans using radioactive substances, by comprising a detecting agent of a B cell receptor (BCR) clonotype specific to an Alzheimer's disease patient.

### CROSS-REFERENCE TO RELATED APPLICATION

This application is a continuation-in-part of application Ser. No. 18/827,742 filed Sep. 7, 2024, which claims priority to Korean Patent Application No. 10-2023-0118789 filed on Sep. 7, 2023, the entire contents of each of which is incorporated herein by reference.

### INCORPORATION BY REFERENCE STATEMENT OF THE MATERIAL IN THE SEQUENCE LISTING XML FILE

The XML file submitted herewith is incorporated by reference in the Specification. The XML file is identified as follows: (i) Name of the File: OF24P125US_sequence list; (ii) Date of Creation: Jan. 22, 2025; (iii) Size of File: 33,390,077 bytes.

### TECHNICAL FIELD

The present specification discloses a composition, a system, and a method for predicting or diagnosing Alzheimer's disease, comprising a detecting agent of a B cell receptor clonotype specific to an Alzheimer's disease patient.

[National Research and Development Project Supporting This Invention]

[Project Identification Number] 1465039792

[Project Number] HU20C0339000023

[Name of Department] Ministry of Health and Welfare

[Name of Project Management (Professional) Institution] Korea Health Industry Development Institute (KHIDI)

[Research Business Name] Dementia Research and Development Program (2023-KHIDI-4635)

[Research Project Title] Investigation of Antibody-Mediated Pathogenesis through B Cell Receptor Repertoire Analysis in Alzheimer's Disease Patients

[Contribution Rate] 1/1

[Name of Project Performing Organization] Seoul National University Research & Development Business Foundation

[Research Period] Apr. 1, 2023 to May 31, 2023

### BACKGROUND

Alzheimer's disease (AD) is a progressive neurodegenerative disorder that causes dementia, cognitive decline, memory loss and impaired daily functioning. The major pathological hallmark of AD is the presence of plaques of the amyloid beta peptide (Aβ) and neurofibrillary tangles (NFTs) of the phosphorylated tau protein1, which are accompanied by neuroinflammation in the central nerve system (CNS). Recent studies have revealed that the pathogenesis of AD is not solely restricted to these changes in the brain. There is mounting evidence that the peripheral immune system is intimately linked to AD pathology, particularly emphasizing the crucial involvement of immune cells in the development of AD. However, there has been minimal discourse concerning the contribution of T and B lymphocytes in AD. Several studies have demonstrated that peripheral homeostasis of T lymphocyte is changed in AD patients, whereas little is known about the role of peripheral B lymphocytes.

Meanwhile, to date, there are few diagnostic tools in the clinical field of AD [5,6]. Although several alternative methods have been developed, people suspected of AD have to rely on several factors such as medical history, neuroimaging data from magnetic resonance imaging (MRI) or position emission tomography (PET), and neuropsychological examination. There are still shortcomings and concerns regarding the molecular accuracy of MRI and PET scans using radioactive substances. In particular. PET imaging, which is considered a first-line diagnostic method based on AD-specific pathological features such as decreased synaptic activity and accumulation of amyloid plaques in the brain, has several considerations that should be carefully considered. It takes time for brain Aβ to accumulate to a certain level of SUV [7,8]. In addition, additional time is needed until pathological symptoms develop and translate into cognitive changes in daily life [9]. In particular, phosphorylated tau protein has been considered a subsequent consequence after the appearance of Δβ, but it appears with a relatively long time interval of about 5 years[10]. Most blood biomarker tests have detected AD-specific pathologic proteins in human blood, serum, and CSF [11,12].

In other words, the current diagnosis of Alzheimer's disease relies on the patient's medical history, brain imaging data, and neuropsychological scale evaluation, and with the development of molecular imaging technology, the presence or absence of medial temporal lobe atrophy and ventricular enlargement due to neurodegeneration can be diagnosed with structural brain imaging tests such as magnetic resonance imaging (MRI) and computed tomography (CT). In addition, the presence or absence of brain function decline and the area of decline can be visualized and confirmed through functional brain imaging tests such as PET (position emission tomography) and SPECT (single-photon emission computed tomography) using fluoro-deoxy-D-glucose (FDG), amyloid tracer, and tau ligand (Cold Spring Harb Perspect Med. 2012 April; 2 (4): a006213, Hum Brain Mapp. 2019 Dec. 15; 40 (18): 5424-5442).

However, structural brain imaging has the disadvantage of low molecular accuracy and cannot detect histopathological features such as amyloid neuritic plaques and neurofibrillary tangles specific to Alzheimer's disease. Functional brain imaging based on synaptic activity and the pathogenesis of Alzheimer's disease (amyloid hypothesis) has relatively high diagnostic accuracy, but there are high screening costs and risks of exposure to radioactive drugs in the human body through intravenous injection. Additionally, FDG residual amount in the brain acts as a nonspecific indicator of metabolic processes, so there is a possibility that it may decrease due to various causes such as ischemia and inflammation unrelated to Alzheimer's disease (Alzheimers Dement. 2018 November; 14 (11): 1522-1552).

In addition, it takes an average of more than 15 years for the standardized uptake values (SUV) of Aβ measured through FDG-PET to reach the Alzheimer's disease-specific threshold value (Neurology. 2013 Mar. 5; 80 (10): 890-6, JAMA. 2017 Jun. 13; 317 (22): 2305-2316), and hyperphosphorylated tau gradually accumulates and spreads together with amyloid neuritic plaques as the clinical stage of the disease progresses (J Neurosci. 2018 May 9; 38 (19): 4482-4489), making it unsuitable for early diagnosis of the disease (Neurology. 2021 Mar. 2; 96 (9): e1347-e1357).

Therefore, in addition to traditional biomarkers, there is a need to explore various targets that can be used for early diagnosis (Front Immunol. 2022; 13:1010946), and there is a growing need for biomarkers that can be detected in a cost-effective and non-invasive manner.

It is believed that blood-based biomarkers are likely to meet these needs, and attempts are actively being made to use them as alternative diagnostic methods by measuring the ratio of Aβ42 and Aβ40 present in the patient's blood, the concentration of tau phosphorylated with specific amino acids, and neurofilament light chain (NfL)-related biomarkers of neurodegeneration (Lancet Neurol. 2022 January; 21 (1): 66-77, J Clin Neurol. 2022 July; 18 (4): 401-409).

Currently, there are three blood-based diagnostic tests for Alzheimer's disease approved by the FDA, but there are differing opinions in the academic community that question the efficacy and necessity of blood-based biomarkers due to the lack of standardized measurement techniques, lack of cutoff values for individual biomarkers, and lack of evaluation of confounding factors such as aging, underlying diseases, and racial differences (Biomark Insights. 2020 Aug. 21; 15:1177271920950319). In addition, blood-based tests cannot be used as a standalone test for diagnosing Alzheimer's disease, and the clinical validity of blood biomarkers can be proven only when the presence and ratio of the corresponding biomarker in the patient's cerebrospinal fluid (CSF) corresponds to the results in blood and also matches the results of existing molecular brain imaging tests.

Accordingly, the present inventors completed the present invention by discovering a BCR clonotype group that is not present in a normal human cohort but is found only in a group of Alzheimer's disease patients through research under the assumption that Alzheimer's disease patients have various B cell receptors associated with the progression or suppression of the disease.

### DETAILED DESCRIPTION

The present inventors confirmed that AD patients share stereotypic BCR clonotypes that are not found in the CG (control group) and that the degree of overlap between patient pairs is higher than that between CG pairs.

Specifically, the present inventors confirmed that the BCR repertoire of AD patients has unique characteristics, and then discovered disease-specific sequences that can be utilized to discover antibodies that bind to pathological markers, and identified 3986 unique BCR clonotypes that are specific only to the AD patient cohort. In addition, the present inventors selected the most prominent clonotypes in terms of degree of sharing and persistence within time points, and confirmed that they have affinity for the Aβ42 peptide, one of the major Δβ peptide isoforms found in amyloid plaques in the brain with AD and reported as an autoantigen. Accordingly, the present inventors confirmed that AD can be predicted or diagnosed by detecting the Alzheimer's-specific B cell receptor (BCR) clonotype.

Therefore, in one aspect, an object of the present invention is to provide a composition, a system, and a method for providing information for predicting or diagnosing Alzheimer's disease.

### Solution to Problem

In one aspect, the present invention provides a composition for predicting or diagnosing Alzheimer's disease, comprising a detecting agent of a B cell receptor (BCR) clonotype specific to an Alzheimer's disease patient in a sample of a subject, wherein the BCR clonotype comprises the V gene; and the J gene; and an amino acid sequence of complementary determining region 3 (CDR3) of the heavy chain of the BCR.

In other aspect, the present invention provides a system for predicting or diagnosing Alzheimer's disease comprising the composition for predicting or diagnosing Alzheimer's disease.

In another aspect, the present invention provides a method for providing information for predicting or diagnosing Alzheimer's disease comprising: detecting a B cell receptor (BCR) clonotype specific to an Alzheimer's disease patient in a sample of a subject, wherein the BCR clonotype comprises the V gene; and the J gene; and an amino acid sequence of complementary determining region 3 (CDR3) of the heavy chain of the BCR.

### Advantageous Effects of Invention

A composition according to one aspect of the present invention has an excellent effect in predicting or diagnosing Alzheimer's disease by simply using peripheral blood mononuclear cells isolated from a subject, in addition to methods such as MRI and PET scans using radioactive substances, by comprising a detecting agent of a B cell receptor (BCR) clonotype specific to an Alzheimer's disease patient.

### BRIEF DESCRIPTION OF THE DRAWINGS

FIGS. 1A to IC are schematic diagrams illustrating a sample acquisition schedule and BCR repertoire analysis pipeline according to one example of the present invention. FIG. 1A shows the peripheral blood samples of 44 patients from 2 different time points were collected for the Alzheimer's disease cohort (AD). The second blood samples from this cohort are chronologically separated by 13.3 months maximum and 3.4 months minimum. The black arrowheads represent the exact sampling points of the samples in the given time frame. FIG. 1B shows the vaccinated control group (CG) comprises of 55 vaccine recipients who were subjected to peripheral blood sampling before the first shot (TP1), 1 week after the 1st shot (TP2), 1 week after the 2nd shot (TP3), 1 month after the 2nd shot (TP4), 6 months after the 2nd shot (TP5), and 1 month after the 3rd shot (TP6). FIG. 1C show the process of performing sequence filtering based on the shown flow chart to obtain BCR clonotypes that are representative of each cohort for further analyses. Clonotypes which were comprised fully of naive sequences were removed and cohort unique clonotypes were obtained. Next, clonotypes that were present in two or more subjects in a cohort were selected. Due to the potential risk of sequence contamination during NGS library prep and during sequencing, clonotypes where patients did not possess unique nucleotide sequences and were completely eclipsed by other patients were ruled out.

FIGS. 2A to 2E are diagrams showing the results of analysis according to one embodiment of the present invention, wherein Alzheimer patients show distinctly high similarity within the AD cohort in context of stereotypic and group specific BCR clonotypes. FIG. 2A is a heat map of the BCR similarity values between each subject pair within each group and time point, and the similarity is calculated as the cosine similarity between two one-hot encoded linear vectors representing the presence (1) or absence (0) of group specific and shared clonotypes within each subject's BCR repertoire. The collection period is denoted inside parentheses. FIGS. 2B and 2C are the boxplots representing the distribution of BCR similarity for all subject pairs of the group and the heat map representing the P-values between them. The dashed line represents the median value for the CG TP1 repertoires. The color of the heat maps show whether the median value of the group written on the y axis is larger (red) or smaller (blue) while the intensity of said colors is encoded by the P-value. FIG. 2D is a Chord plot displaying the similarity matrix of the top 20 AD patient pairs. The similarities were calculated from the combined repertoires of both time points if they existed. The chord width represents the similarity value with the thickest chord connecting patient 26 and 28 represent the value 0.961 while the thinnest chord connecting patients 11 and 25 represents a value of 0.063. FIG. 2E is a diagram showing the number of shared clonotypes between each AD patient pairs for the time point combined repertoires, and the pair with the most number of shared clonotypes is denoted with a red star pattern.

FIGS. 3A to 3E are diagrams showing the results of an in vitro reactivity experiment for Aβ42 peptide according to one embodiment of the present invention, wherein the in vitro experimental results show highly shared BCR sequences within the Alzheimer patient cohort weakly bind to Aβ42 peptide. FIG. 3A is a table displaying the AD group specific BCR clonotypes that are shared by at least 4 different patients. The grey columns represent samples omitted from the TP2 stage due to dropouts. Patients who possess similar clonotypes, 1 amino acid mismatch in the CDR3 sequence, are also shown in the table and are coded with lighter colors. FIG. 3B is a graph showing the number of CG repertoires that possess similar clonotypes for the AD specific clonotypes shown in FIG. 3A. FIG. 3C shows amino acid sequence alignment and representation of highly shared and highly persistent BCR sequence. FIG. 3D shows the light chain sequence information for the Aβ42 binders found via biopanning, A-4-3 and A-4-52. FIG. 3E is ELISA results of recombinant scFv of A-4-3 and A-4-52 clones against Aβ42, and the graph is normalized by the absorbance difference between Aβ42 and BSA (blocking only) signal.

FIGS. 4A to 4C are graphs showing the results of analyzing the similarity of Alzheimer's-specific-stereotypic BCR clonotypes according to one embodiment of the present invention, wherein Alzheimer patients show distinctly high similarity within the AD cohort in context of stereotypic BCR clonotypes even without filtering by the control group. FIG. 4A is a heat map of the BCR similarity values between each subject pair within each group and time point. The similarity is calculated as the cosine similarity between two one-hot encoded linear vectors representing the presence (1) or absence (0) of shared and not group specific clonotypes within each subject's BCR repertoire. The collection period is denoted inside parentheses. FIGS. 4B and 4C are the boxplots representing the distribution of BCR similarity for all subject pairs of the group and the heat map representing the P-values between each group. The dashed line represents the median value for the CG TP1 repertoires. The color of the heat maps shows whether the median value of the cohort written on the y axis is larger (red) or smaller (blue) while the intensity of said colors is encoded by the P-value.

FIG. 5 is a diagram showing the experimental results confirming the expression of scFv-hFc fusion proteins according to one embodiment of the present invention. Total six out of eight Aβ42-binding scFv clones were successfully expressed in mammalian expression system. All recombinant scFv proteins tagged with human IgG1 FC (hFc), a. Representation of scFv-hFc fusion protein. The expected protein size is 56 kDa in non-reducing state (NR) and double in reducing condition (R) because of the dimerization via disulfide bond with cysteine residue in hFc tag.

Hereinafter, the present invention will be described in detail.

In one aspect of the present invention, the “Alzheimer prediction” may refer to predicting or diagnosing whether a patient has a risk for Alzheimer, whether the risk for Alzheimer is relatively high, what the cause of Alzheimer is, or whether Alzheimer has already occurred. Additionally, in one aspect of the present invention, the “Alzheimer diagnosis” may refer to confirming presence or features of pathological conditions. On the purpose of one aspect of the present invention, diagnosis may refer to confirming whether Alzheimer has occurred. The composition, kit or method according to one aspect of the present invention may be used to delay the onset or prevent the occurrence of Alzheimer through special and appropriate care for a specific patient, who is a patient having a high risk of developing Alzheimer. In addition, the composition, kit or method according to one aspect of the present invention may be clinically used to determine treatment by selecting the most appropriate treatment method through early diagnosis of Alzheimer.

In one aspect of the present invention, the “BCR clonotype” may comprise an amino acid sequence of a V gene; a J gene; and a complementary determining region 3 (CDR3) of a heavy chain of a B cell receptor (BCR), or may consist of an amino acid sequence of a V gene; and a J gene; and a CDR3 of a heavy chain of a BCR, or may be a combination of an amino acid sequence of a V gene; and a J gene; and a CDR3 of a heavy chain of a BCR.

In one aspect of the present invention, “detecting a BCR clonotype” may be detecting at least one selected from the group consisting of the following a), b), and c):
a) detecting at least one of the V gene type of the BCR heavy chain, a base sequence of the V gene, and an amino acid sequence encoded from the base sequence of the V gene; at least one of the J gene type of the BCR heavy chain, a base sequence of the J gene, and an amino acid sequence encoded from the base sequence of the J gene; and at least one of the base sequence and amino acid sequence of CDR3 of the BCR heavy chain;
b) detecting the entire base sequence of the BCR clonotype comprising the specific base sequence of the V gene of the BCR heavy chain; the specific base sequence of the J gene of the BCR heavy chain; and the base sequence of the CDR3 of the BCR heavy chain; and
c) detecting the entire amino acid sequence of the BCR clonotype comprising the specific amino acid sequence encoded from the V gene of the BCR heavy chain; the specific amino acid sequence encoded from the J gene of the BCR heavy chain; and the amino acid sequence of the CDR3 of the BCR heavy chain.

For example, detecting a BCR clonotype from a sample of a subject may be detecting a BCR clonotype in which the V gene of the heavy chain of the BCR is IGHV1-18, the J gene is IGHJ1, and the amino acid sequence of the CDR3 is SEQ ID NO: 72 from the sample of the subject, or may be detecting the entire base sequence of the BCR clonotype including a specific base sequence encoding a specific base sequence of the IGHV1-18, a specific base sequence of the IGHJ1, and a base sequence encoding an amino acid sequence of the CDR3 represented by SEQ ID NO: 72, or may be detecting the entire amino acid sequence of the BCR clonotype including a specific amino acid sequence encoded by the IGHV1-18, a specific amino acid sequence encoded by the IGHJ1, and an amino acid sequence of the CDR3 represented by SEQ ID NO: 72.

In one aspect of the present invention, “homology of 80 to 100%” may mean that the homology is 80% or more, 81% or more, 82% or more, 83% or more, 84% or more, 85% of more, 86% or more, 87% or more, 88% or more, 89% or more, 90% or more, 91% or more, 92% or more, 93% or more, 94% or more, 95% or more, 96% or more, 97% or more, 98% or more, or 99% or more, and the homology is 100% or less, 99% or less, 98% or less, 97% or less, 96% or less. 95% or less. 94% or less, 93% or less, 92% or less, 91% or less, 90% or less, 89% or less, 88% or less, 87% or less, 86% or less, 85% or less, It could mean 84% or less, 83% or less, 82% or less, or 81% or less.

In one aspect, the present invention provides a composition for predicting or diagnosing Alzheimer's disease, comprising a detecting agent of a B cell receptor (BCR) clonotype specific to an Alzheimer's disease patient in a sample of a subject, wherein the BCR clonotype comprises the V gene; and the J gene; and an amino acid sequence of complementary determining region 3 (CDR3) of the heavy chain of the BCR.

The composition according to one aspect of the present invention may comprise a detecting agent of a B cell receptor (BCR) clonotype specific to an Alzheimer's disease patient. A composition according to one aspect of the present invention may predict or diagnose a subject as having a BCR clonotype specific to an Alzheimer's patient, and specifically may predict or diagnose the subject as having Alzheimer's disease, if a BCR clonotype specific to an Alzheimer's patient is detected in a sample of the subject.

The composition according to one aspect of the present invention may predict or diagnose the subject as having Alzheimer's disease when the BCR clonotype detected from the sample of the subject is i) the V gene is at least one selected from the group consisting of IGHV (Immunoglobulin Heavy Variable) 1-18, IGHV1-2, IGHV1-24, IGHV1-3, IGHV1-46, IGHV1-58, IGHV1-69, IGHV1-8, IGHV2-26, IGHV2-5, IGHV2-70, IGHV3-11, IGHV3-13, IGHV3-15, IGHV3-20, IGHV3-21, IGHV3-23, IGHV3-30, IGHV3-30-3, IGHV3-33, IGHV3-43, IGHV3-43D, IGHV3-48, IGHV3-49, IGHV3-53, IGHV3-64, IGHV3-64D, IGHV3-66, IGHV3-7, IGHV3-72, IGHV3-73, IGHV3-74, IGHV3-9, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, and IGHV7-4-1, ii) the J gene in the BCR clonotype detected from a sample of the subject is at least one selected from the group consisting of IGHJ (Immunoglobulin Heavy Joining) 1, IGHJ2, IGHJ3, IGHJ4, IGHJ5, and IGHJ6, and iii) the amino acid sequence of CDR3 in the BCR clonotype detected from a sample of the subject is 80 to 100% homologous to at least one selected from the group consisting of SEQ ID NOs: 72 to 4047, and 32396 to 32481.

The composition according to one aspect of the present invention may predict or diagnose the subject as having Alzheimer's disease when the amino acid sequence of CDR3 in the BCR clonotype detected from a sample of the subject is 80 to 100% homologous to at least one selected from the group consisting of SEQ ID NOs: 72 to 4047, and 32396 to 32481. Specifically, the composition may predict or diagnose the subject as having Alzheimer's disease when the amino acid sequence of CDR3 in the BCR clonotype detected from a sample of the subject is 80 to 100% homologous to at least one selected from the group consisting of ARDLPATGAFDI (SEQ ID NO: 214), VRLAEYFQN (SEQ ID NO: 875), TRDRRGWD (SEQ ID NO: 1211), ALGRGMDV (SEQ ID NO: 1048), VLRGSAFDI (SEQ ID NO: 798), ARSGGLDP (SEQ ID NO: 668), AGGRSFDY (SEQ ID NO: 613), AGNLMDV (SEQ ID NO: 2257), ARRGEY (SEQ ID NO: 2070), ARDMFRGIPDYLDY (SEQ ID NO: 1988), AKSLGSGTYSFDY (SEQ ID NO: 1326), ARNAGLDY (SEQ ID NO: 1128), ARDDGYRSIDY (SEQ ID NO: 990), VRGGGSGWPFES (SEQ ID NO: 3709), SRGRDGIG (SEQ ID NO: 2815), ARSGGLDV (SEQ ID NO: 2415), ARSRSGSYYYGMDV (SEQ ID NO: 1198), AGDWNDDDIFDY (SEQ ID NO: 32396), ARKGEQLWHY (SEQ ID NO: 965), and ARDPIAVPGLFDY (SEQ ID NO: 347).

The composition according to one aspect of the present invention may predict or diagnose the subject as having Alzheimer's disease when the BCR clonotype detected from the sample of the subject is at least one of BCR clonotypes of the V gene; the J gene; and the amino acid sequence of CDR3.

For example, the composition according to one aspect of the present invention may predict or diagnose the subject as having Alzheimer's disease when the V gene and J gene in the BCR clonotype detected from the sample of the subject are IGHV3-7 and IGHJ1, and the amino acid sequence of CDR3 is the amino acid sequence of SEQ ID NO: 875.

The composition according to one aspect of the present invention may predict or diagnose the subject as having Alzheimer's disease when the detecting agent of a BCR clonotype according to one aspect of the present invention is a detecting agent of the entire amino acid sequence of the BCR clonotype, comprising an amino acid sequence encoded from the V gene of the heavy chain of the BCR; an amino acid sequence encoded from the J gene of the heavy chain of the BCR; and an amino acid sequence of CDR3 of the heavy chain of the BCR and the entire amino acid sequence of the BCR clonotype detected from the sample of the subject is at least one of SEQ ID NOs: 18222 to 32395. For example, the composition according to one aspect of the present invention may predict or diagnose the subject as having Alzheimer's disease when the entire amino acid sequence of the BCR clonotype detected from the sample of the subject is SEQ ID NO: 21427.

The composition according to one aspect of the present invention may predict or diagnose the subject as having Alzheimer's disease when the detecting agent of a BCR clonotype according to one aspect of the present invention is a detecting agent of the entire base sequence of the BCR clonotype, comprising a base sequence of the V gene of the heavy chain of the BCR; a base sequence of the J gene of the heavy chain of the BCR; and a base sequence encoding an amino acid sequence of CDR3 of the heavy chain of the BCR and the entire base sequence of the BCR clonotype detected from the sample of the subject is at least one of SEQ ID NOs: 4048 to 18221. For example, the composition according to one aspect of the present invention may predict or diagnose the subject as having Alzheimer's disease when the entire base sequence of the BCR clonotype detected from the sample of the subject is SEQ ID NO: 7253.

The composition according to one aspect of the present invention may predict or diagnose the subject as having Alzheimer's disease when the BCR clonotype detected from the sample of the subject is at least one of BCR clonotypes of the V gene; the J gene; and the amino acid sequence of CDR3.

The detecting agent according to one aspect of the present invention may comprise a detecting agent of a protein specific to the BCR clonotype or a detecting agent of a nucleic acid encoding the protein.

For example, the detecting agent may comprise the below A) or B):
A) a protein detecting agent capable of detecting at least one selected from the group consisting of an amino acid sequence encoded by a base sequence of a V gene included in the BCR clonotype; an amino acid sequence encoded by a base sequence of a J gene included in the BCR clonotype; an amino acid sequence of CDR3; and an entire amino acid sequence of a BCR clonotype including a specific amino acid sequence encoded by the V gene, a specific amino acid sequence encoded by the J gene, and an amino acid sequence of CDR3; or
B) a detection agent capable of detecting at least one selected from the group consisting of a base sequence of a V gene included in the BCR clonotype; a base sequence of a J gene included in the BCR clonotype; a base sequence of a CDR3; and an entire base sequence of a BCR clonotype including a specific base sequence of the V gene, a specific base sequence of the J gene, and a base sequence of CDR3.

The detecting agent of a protein according to one aspect of the present invention may comprise an antibody or aptamer specific to the BCR clonotype.

Specifically, the antibody may be one or more selected from the group consisting of a polyclonal antibody, a monoclonal antibody, a recombinant antibody, and a combination thereof. More specifically, the antibody may include all of not only the polyclonal antibody, the monoclonal antibody, the recombinant antibody, and a complete form having two light chains with the full length and two heavy chains with the full length, but also functional fragments of the antibody molecules, for example, Fab, F(ab′), F(ab′)2, and Fv. The antibodies may be easily prepared by using a well-known technique in the art and antibodies which are prepared and commercially sold may be used.

In addition, specifically, the aptamer refers to a single-stranded oligonucleotide, and refers to a nucleic acid molecule having binding activity to a predetermined target molecule. The aptamer may have various three-dimensional structures depending on its base sequence, and may have high affinity for a specific substance, such as an antigen-antibody reaction. The aptamer may inhibit the activity of a predetermined target molecule by binding to the predetermined target molecule. The aptamer of the present invention according to one aspect of the present invention may be RNA, DNA, a modified nucleic acid, or a mixture thereof, and may have a linear or cyclic shape, but is not limited thereto. The aptamer according to one aspect of the present invention may be used for the BCR clonotype. The aptamer having binding activity to the protein may be easily produced by a person skilled in the art by referring to each base sequence according to a known method.

The detecting agent of a nucleic acid encoding the protein specific for a BCR clonotype according to one aspect of the present invention may comprise at least one selected from the group consisting of antisense oligonucleotide, primer pair, probe, and polynucleotide that specifically binds to mRNA or DNA encoding the BCR clonotype. That is, detection of a nucleic acid may be performed by an amplification reaction using one or more oligonucleotide primer that hybridizes to a nucleic acid molecule encoding a gene or a complement of the nucleic acid molecule. For example, the detection of a nucleic acid using a primer may be performed by amplifying a gene sequence using an amplification method such as PCR, and then confirming whether the gene is amplified by a method known in the art.

In one aspect of the present invention, the “primer” refers to a polynucleotide having a base sequence that can complementarily bind to the end of a specific region of a gene, or a mutant thereof, which is used for amplifying the specific region corresponding to a target region of the gene by PCR. The primer is not required to be completely complementary to the end of the specific region, and may be used as long as it is complementary to the end to such an extent that it can form a double-stranded structure by hybridizing with the end.

In one aspect of the present invention, the “probe” refers to a polynucleotide having a base sequence that can complementarily bind to a target of a gene, a mutant thereof, or a polynucleotide and a labeling substance bound thereto.

In one aspect of the present invention, “hybridization” means that 2 single-stranded nucleic acids form a duplex structure by pairing complementary base sequences. Hybridization may occur not only when there is complete complementary pairing between single-stranded nucleic acid sequences (perfect match), but also when there are partially mismatched (mismatch) bases.

The composition according to one aspect of the present invention may further comprise labels which may quantitatively or qualitatively measure formation of an antigen-antibody complex, general tools used in the immunological analysis, reagents, and the like as well as the detecting agent of a protein specific to the BCR clonotype or the detecting agent of a nucleic acid encoding the protein.

In one aspect of the present invention, the labels which may quantitatively or qualitatively measure the formation of the antigen-antibody complex include enzymes, fluorescent substances, ligands, luminescent substances, microparticles, redox molecules, radioactive isotopes, and the like, and are not necessarily limited thereto. The enzymes usable as the detection label include β-glucuronidase, β-glucosidase, β-galactosidase, urease, peroxidase, alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, β-lactamase, and the like, and are not limited thereto. The fluorescent substances include fluorescein, isothiocyanate, rhodamine, phycoerythrin, phycocyanin, allophycocyanin, o-phthaldehyde, fluorescamine, and the like, and are not limited thereto. The ligands include biotin derivatives and the like, and are not limited thereto. The luminescent substances include acridinium ester, luciferin, luciferase, and the like, and are not limited thereto. The microparticles include colloidal gold, colored latex, and the like, and are not limited thereto. The redox molecules include ferrocene, ruthenium complex compounds, viologen, quinone, Ti ions, Cs ions, diimide, 1,4-benzoquinone, hydroquinone, K₄W(CN)⁸, [Os(bpy)₃]²⁺, [RU(bpy)₃]²⁺, [MO(CN)₈]⁴⁻, and the like, and are not limited thereto. The radioactive isotopes include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, ¹⁸⁶Re, and the like, and are not limited thereto.

In one aspect of the present invention, an example of the tool or the reagent includes suitable carriers, solubilizing agents, detergents, buffering agents, stabilizers, and the like, but is not limited thereto. When the marker is the enzyme, a substance and a quencher which may measure the enzyme activity may be included. The carriers include a soluble carrier, and an insoluble carrier. An example of the soluble carrier includes a buffer solution that is physiologically acceptable and known in the art, for example, PBS, and an example of the insoluble carrier may include polystyrene, polyethylene, polypropylene, polyester, polyacrylonitrile, fluororesin, cross-linked dextran, polysaccharide, other papers, glass, metal, agarose, and a combination thereof.

The composition according to one aspect of the present invention may be applied to a sample of a subject, and the sample refers to all samples obtained from an individual from which a protein detection agent specific for a B cell receptor (BCR) clonotype specific for an Alzheimer's patient or a nucleic acid encoding the protein according to one aspect of the present invention may be detected. Specifically, the sample may be one or more selected from the group consisting of saliva, biopsy, blood, skin tissue, liquid culture, feces and urine, but is not limited thereto, and may be treated and prepared by a method which is generally used in the art.

In other aspect, the present invention provides a system for predicting or diagnosing Alzheimer's disease comprising the composition for predicting or diagnosing Alzheimer's disease. The description of the Alzheimer's disease, the B cell receptor (BCR) clonotype specific to the Alzheimer's patient, the detecting agent, and the prediction or diagnosis of Alzheimer's is as described above.

The system according to one aspect of the present invention may further comprise an instruction.

The instruction according to one aspect of the present invention may describe that when the BCR clonotype specific to the Alzheimer's patient is detected in the sample of the subject, the subject is predicted or diagnosed as having the BCR clonotype specific to the Alzheimer's patient, and specifically, the subject is predicted or diagnosed as having Alzheimer's.

The system according to one aspect of the present invention may be applied to a sample of a subject, and the sample refers to all samples obtained from an individual from which a protein detection agent specific for a B cell receptor (BCR) clonotype specific for an Alzheimer's patient or a nucleic acid encoding the protein according to one aspect of the present invention may be detected. Specifically, the sample may be one or more selected from the group consisting of saliva, biopsy, blood, skin tissue, liquid culture, feces and urine, but is not limited thereto, and may be treated and prepared by a method which is generally used in the art.

In another aspect, the present invention provides a method for providing information for predicting or diagnosing Alzheimer's disease comprising: detecting a B cell receptor (BCR) clonotype specific to an Alzheimer's disease patient in a sample of a subject, wherein the BCR clonotype comprises the V gene; and the J gene; and an amino acid sequence of complementary determining region 3 (CDR3) of the heavy chain of the BCR. The description of the Alzheimer's disease, the B cell receptor (BCR) clonotype specific to the Alzheimer's patient, the detecting agent, and the prediction or diagnosis of Alzheimer's is as described above.

The method for providing information according to one aspect of the present invention may further comprise predicting or diagnosing the subject as having Alzheimer's disease when the BCR clonotype detected from the sample of the subject is i) the V gene is at least one selected from the group consisting of IGHV (Immunoglobulin Heavy Variable) 1-18, IGHV1-2, IGHV1-24, IGHV1-3, IGHV1-46, IGHV1-58, IGHV1-69, IGHV1-8, IGHV2-26, IGHV2-5, IGHV2-70, IGHV3-11, IGHV3-13, IGHV3-15, IGHV3-20, IGHV3-21, IGHV3-23, IGHV3-30, IGHV3-30-3, IGHV3-33, IGHV3-43, IGHV3-43D, IGHV3-48, IGHV3-49, IGHV3-53, IGHV3-64, IGHV3-64D, IGHV3-66, IGHV3-7, IGHV3-72, IGHV3-73, IGHV3-74, IGHV3-9, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, and IGHV7-4-1, ii) the J gene in the BCR clonotype detected from a sample of the subject is at least one selected from the group consisting of IGHJ (Immunoglobulin Heavy Joining) 1, IGHJ2, IGHJ3, IGHJ4, IGHJ5, and IGHJ6, and iii) the amino acid sequence of CDR3 in the BCR clonotype detected from a sample of the subject is 80 to 100% homologous to at least one selected from the group consisting of SEQ ID NOs: 72 to 4047, and 32396 to 32481.

The method for providing information according to one aspect of the present invention may further comprise predicting or diagnosing the subject as having Alzheimer's disease when the BCR clonotype detected from the sample of the subject is at least one of BCR clonotypes of the V gene; the J gene; and the amino acid sequence of CDR3. For example, the method for providing information according to one aspect of the present invention may further comprise predicting or diagnosing the subject as having Alzheimer's disease when the V gene and J gene in the BCR clonotype detected from the sample of the subject are IGHV3-7 and IGHJ1, and the amino acid sequence of CDR3 is the amino acid sequence of SEQ ID NO: 875.

The method for providing information according to one aspect of the present invention may further predicting or diagnosing the subject as having Alzheimer's disease when the entire amino acid sequence of the BCR clonotype detected from the sample of the subject comprising an amino acid sequence encoded from the V gene of the heavy chain of the BCR; an amino acid sequence encoded from the J gene of the heavy chain of the BCR; and an amino acid sequence of CDR3 of the heavy chain of the BCR is at least one of SEQ ID NOs: 18222 to 32395. For example, the method for providing information according to one aspect of the present invention may further comprise predicting or diagnosing the subject as having Alzheimer's disease when the entire amino acid sequence of the BCR clonotype detected from the sample of the subject is SEQ ID NO: 21427.

The method for providing information according to one aspect of the present invention may further predicting or diagnosing the subject as having Alzheimer's disease when the entire base sequence of the BCR clonotype detected from the sample of the subject comprising a base sequence of the V gene of the heavy chain of the BCR; a base sequence of the J gene of the heavy chain of the BCR; and a base sequence encoding an amino acid sequence of CDR3 of the heavy chain of the BCR is at least one of SEQ ID NOs: 4048 to 18221. For example, the method for providing information according to one aspect of the present invention may further comprise predicting or diagnosing the subject as having Alzheimer's disease when the entire base sequence of the BCR clonotype detected from the sample of the subject is SEQ ID NO: 7253.

The method for providing information according to one aspect of the present invention may further predicting or diagnosing the subject as having Alzheimer's disease when the amino acid sequence of CDR3 in the BCR clonotype detected from a sample of the subject is 80 to 100% homologous to at least one selected from the group consisting of SEQ ID NOs: 72 to 4047, and 32396 to 32481. Specifically, the method for providing information may comprise predicting or diagnosing the subject as having Alzheimer's disease when the amino acid sequence of CDR3 in the BCR clonotype detected from a sample of the subject is 80 to 100% homologous to at least one selected from the group consisting of ARDLPATGAFDI (SEQ ID NO: 214), VRLAEYFQN (SEQ ID NO: 875), TRDRRGWD (SEQ ID NO: 1211), ALGRGMDV (SEQ ID NO: 1048), VLRGSAFDI (SEQ ID NO: 798), ARSGGLDP (SEQ ID NO: 668), AGGRSFDY (SEQ ID NO: 613), AGNLMDV (SEQ ID NO: 2257), ARRGEY (SEQ ID NO: 2070), ARDMFRGIPDYLDY (SEQ ID NO: 1988), AKSLGSGTYSFDY (SEQ ID NO: 1326), ARNAGLDY (SEQ ID NO: 1128), ARDDGYRSIDY (SEQ ID NO: 990), VRGGGSGWPFES (SEQ ID NO: 3709), SRGRDGIG (SEQ ID NO: 2815), ARSGGLDV (SEQ ID NO: 2415), ARSRSGSYYYGMDV (SEQ ID NO: 1198), AGDWNDDDIFDY (SEQ ID NO: 32396), ARKGEQLWHY (SEQ ID NO: 965), and ARDPIAVPGLFDY (SEQ ID NO: 347).

The method for providing information according to one aspect of the present invention may further comprise predicting or diagnosing the subject as having Alzheimer's disease when the BCR clonotype detected from the sample of the subject is at least one of BCR clonotypes of the V gene; the J gene; and the amino acid sequence of CDR3.

The sample of the subject according to one aspect of the present invention may comprise nucleic acid of the subject, wherein the nucleic acid may comprise DNA, mRNA, or cDNA synthesized from mRNA.

Hereinafter, the configuration and effects of the present invention will be described in more detail by way of examples. However, the following examples are for illustrative purposes only and it will be apparent to those of ordinary skill in the art that the scope of the present invention is not limited by the examples.

The present inventors identified BCR clonotypes persistently present and shared among AD patients but not present in normal healthy populations and analyzed their characteristics. For this, we collected peripheral blood (PB) from 44 AD patients at two sampling points with the interval of 14-53 weeks and used them to re-constitute in silico BCR repertoires of these patients. As a control group, the inventors used the BCR repertoire from 55 healthy volunteers vaccinated with SARS-CoV2 mRNA vaccine whose PB samples were collected for six times over 42 weeks 15. In comparison, the AD patients showed much higher sharing of stereotypic patient-specific BCR clonotypes compared to the control group. One of these BCR clonotypes was found to encode antibodies reactive to Aβ42 peptide, which are the well-known antigen of autoantibodies found in AD patients.

### Statistical Analysis

In the below Examples and Experimental examples, the P values were calculated using the student t-test unless specified during comparison of BCR repertoire characteristics. Unless otherwise indicated. P-value <0.05 was regarded as significant.

### Example 1—Blood Sampling and Peripheral Blood Mononuclear Cell Isolation from Study Subjects

The present inventors acquired data of all subjects, including demographic information, subtypes of dementia, Clinical Dementia Rating (CDR), comprehensive neuropsychological tests with Mini-Mental State Examination (MMSE), neuroimaging results by using magnetic resonance imaging (MRI) and amyloid-PET, from the Korean Longitudinal Study on Cognitive Aging and Dementia (KLOSCAD) [16]. Each data was valid for 6 months except amyloid-PET. This study was approved under the recommendations of the Institutional Review Board (IRB) of the Seoul National University Bundang Hospital (SNUBH, IRB approval number, B-2102-664-305), South Korea. Protocols and manuals were also approved by the IRB of the SNUBH.

Total 55 of vaccinees who received BNT162b2 mRNA vaccine were participated, and their peripheral blood sampling was approved by the IRB of Seoul National University Hospital (SNUH, IRB approval number, 2102-032-1193). The details of the vaccination cohort data are described in previous article[1].

Additionally, for the above subjects, all peripheral blood samples were collected by EDTA tubes (BD Vacutainer K2EDTA tubes, 367525) at least 15 ml in total per a patient[17]. Peripheral blood mononuclear cells (PBMCs) and plasma were separated by density-gradient centrifugation using Ficoll (Cytiva Ficoll-Paque Plus, 17-1440-02) solution. After the PBMC suspension was resuspended in CELLBANKER (Amsbio CELLBANKER 2, 11914), the collected PBMCs were immediately cryopreserved and stored in a Freezing container (Nalgene Mr. Frosty, C1562) for a week. Total RNA was isolated by TRIzol Reagent (Invitrogen TRIzol Reagents, 15596026) following the manufacturer's instructions.

### Example 2—Preparation of Next-Generation Sequencing (NGS) Library and NGS Data Processing

An NGS library was prepared using the peripheral blood mononuclear cells of Example 1 above by the following method, and the NGS data obtained therefrom were processed as follows.

Specifically, for the NGS library preparation of BCR heavy chain sequence, genes encoding the variable region of the heavy chain (V_{H}) at the 5 prime forward part and the first constant region of the heavy chain (CH1) domain at the 3 prime reverse part were amplified by specific PCR primers as reported previously[4]. All primer sequences are listed in Tables 1 and 2. In short, one microgram of total RNA was used as the input template for library preparation. Next, reverse transcription was conducted as following the manufacturer's instructions using SuperScript IV First-Strand Synthesis System (Invitrogen, 18091050) with CH1 gene-specific primers for five immunoglobulin heavy chain isotypes containing UMI (unique molecular identifiers) barcodes that consist of 12 random nucleotides. 14 base pair in total; NNNNTNNNNTNNNN, and partial Illumina adapters (No. 1-8). After first-strand cDNA synthesis, it was purified with SPRI beads (Beckman Coulter AMPure XP Reagents, A63882) at a 1:1.8 ratio and second-strand cDNA was synthesized using a KAPA Biosystems kit (Roche KAPA HiFi Hotstart PCR kit, KK2502) with VH genespecific primers (No. 9-14; 95° C. for 3 min, 98° C. for 30 s, 60° C. for 45 s, and 72° C. for 6 min). Double-stranded cDNA was purified through SPRI beads at a 1:1 ratio.

Following that, it was amplified using KAPA Biosystems kit and double primers containing Illumina adapters and index sequences (No. 15, 16; 95° C. for 3 min, 25 cycles of 98° C. for 30 s, 60° C. for 30 s, 72° C. for 1 min, and 72° C. for 5 min). The PCR products were subjected to electrophoresis on a 1.5% agarose gel to separate through the length of DNA and purified using QIAquick gel extraction kit (QIAGEN, 28704) following the manufacturer's protocols. The final NGS libraries were obtained using SPRI beads at a 1:1 ratio and quantified with a 4200 TapeStation System (Agilent Technologies, G2991BA) for QC. Libraries with a single peak of appropriate length were subjected to NGS analysis using the Illumina Nocaseq6000 platform.

TABLE 1
Primers used in the study
Primer used for the preparation of BCR repertoire

| Primer | | Sequence | |
|---|---|---|---|
| No. | Name | (5′-Primer Seq-3′) | Step |
| 1 | RT_ | TGACTGGAGTTCAGACGTGTGC | cDNA |
| | IgG_ | TCTTCCGATCTNNNNTNNNNTN | synthesis |
| | 1_ | NNNAGCCCAGGGCCGCTGTGC | |
| | 250PE | (SEQ ID NO: 1) | |
| 2 | RT_ | TGACTGGAGTTCAGACGTGTGC | cDNA |
| | IgG_ | TCTTCCGATCTNNNNTNNNNTN | synthesis |
| | 2_ | NNNAGCCCAGGGCTGCTGTGC | |
| | 250PE | (SEQ ID NO: 2) | |
| 3 | RT_ | TGACTGGAGTTCAGACGTGTGC | cDNA |
| | IgG_ | TCTTCCGATCTNNNNTNNNNTN | synthesis |
| | 3_ | NNNAGCCCAGGGCGGCTGTGC | |
| | 250PE | (SEQ ID NO: 3) | |
| 4 | RT_ | TGACTGGAGTTCAGACGTGTGC | cDNA |
| | IgA1_ | TCTTCCGATCTNNNNTNNNNTN | synthesis |
| | 2_ | NNNGCAGGCGATGACCACGTTC | |
| | 50PE | C (SEQ ID NO: 4) | |
| 5 | RT_ | TGACTGGAGTTCAGACGTGTGC | cDNA |
| | IgA2_ | TCTTCCGATCTNNNNINNNNTN | synthesis |
| | 2_ | NNNGCATGCGACGACCACGTTC | |
| | 50PE | C (SEQ ID NO: 5) | |
| 6 | RT_ | TGACTGGAGTTCAGACGTGTGC | cDNA |
| | IgE_ | TCTTCCGATCTNNNNTNNNNTN | synthesis |
| | 250 | NNNGAGTCACGGAGGTGGCATT | |
| | PE | GG (SEQ ID NO: 6) | |
| 7 | RT_ | TGACTGGAGTTCAGACGTGTGC | cDNA |
| | IgM_ | TCTTCCGATCTNNNNTNNNNTN | synthesis |
| | 25 | NNNCAACGGCCACGCTGCTCG | |
| | 0PE | (SEQ ID NO: 7) | |
| 8 | RT_ | TGACTGGAGTTCAGACGTGTGC | cDNA |
| | IgD_ | TCTTCCGATCTNNNNTNNNNTN | synthesis |
| | 25 | NNNATGCCAGGACCACAGGGCT | |
| | 0PE | G (SEQ ID NO: 8) | |
| 9 | PCR_ | ACACTCTTTCCCTACACGACGC | Second- |
| | f_VH1 | TCTTCCGATCTGGCCTCAGTGA | strand |
| | | AGGTCTCCTGCAAG | synthesis |
| | | (SEQ ID NO: 9) | |
| 10 | PCR_ | ACACTCTTTCCCTACACGACGC | Second- |
| | f_VH2 | TCTTCCGATCTGTCTGGTCCTA | strand |
| | | CGCTGGTGAAACCC | synthesis |
| | | (SEQ ID NO: 10) | |
| 11 | PCR_ | ACACTCTTTCCCTACACGACGC | Second- |
| | f_VH3 | TCTTCCGATCTCTGGGGGGTCC | strand |
| | | CTGAGACTCTCCTG | synthesis |
| | | (SEQ ID NO: 11) | |
| 12 | PCR_ | ACACTCTTTCCCTACACGACGC | Second- |
| | f_VH4 | TCTTCCGATCTCTTCGGAGACC | strand |
| | | CTGTCCCTCACCTG | synthesis |
| | | (SEQ ID NO: 12) | |
| 13 | PCR_ | ACACTCTTTCCCTACACGACGC | Second- |
| | f_VH5 | TCTTCCGATCTCGGGGAGTCTC | strand |
| | | TGAAGATCTCCTGT | synthesis |
| | | (SEQ ID NO: 13) | |
| 14 | PCR_ | ACACTCTTTCCCTACACGACGC | Second- |
| | f_VH6 | TCTTCCGATCTTCGCAGACCCT | strand |
| | | CTCACTCACCTGTG | synthesis |
| | | (SEQ ID NO: 14) | |
| 15 | Illumida | AATGATACGGCGACCACCGAGA | Indexing |
| | adaptor | TCTACAC [i5 index] | PCR |
| | amp-fwd | ACACTCTTTCCCTACACGACGC | |
| | | TCTTCCGATC | |
| | | (SEQ ID NO: 15) | |
| 16 | Illumida | CAAGCAGAAGACGGCATACGAG | Indexing |
| | adaptor | AT [i7 index] | PCR |
| | amp-rev | GTGACTGGAGTTCAGACGTGTG | |
| | | CTCTTCCG | |
| | | (SEQ ID NO: 16) | |

TABLE 2
Primers used in the study
Primer used for the construction of human scFv libraries

| Primer | | | |
|---|---|---|---|
| No. | Name | Sequence (5′-Primer Seq-3′) | Step |
| 17 | KC | GGCACACAACAGAGGCAGTTCC (SEQ ID NO: 17) | cDNA |
| | | | synthesis |
| 18 | LC_RT1 | GCTTGAAGCTCCTCAGAGGAGG (SEQ ID NO: 18) | cDNA |
| | | | synthesis |
| 19 | LC_RT2 | GCTTGGAGCTCCTCAGAGGAGG (SEQ ID NO: 19) | cDNA |
| | | | synthesis |
| 20 | LC_RT3 | TGGAGCTCCCTGCCTCTGG (SEQ ID NO: 20) | cDNA |
| | | | synthesis |
| 21 | KV1 | GGGCCCAGGCGGCCGAGCTCCAGATGACCCAGTCTCCATCCTC | Second- |
| | | (SEQ ID NO: 21) | strand |
| | | | synthesis |
| 22 | KV2 | GGGCCCAGGCGGCCGAGCTCGTGATGACTCAGTCTCCACTCTCC | Second- |
| | | (SEQ ID NO: 22) | strand |
| | | | synthesis |
| 23 | KV3 | GGGCCCAGGCGGCCGAGCTCGTGTTGACACAGTCTCCAGCC | Second- |
| | | (SEQ ID NO: 23) | strand |
| | | | synthesis |
| 24 | KV4 | GGGCCCAGGCGGCCGAGCTCGTGATGACCCAGACTCCACTCTC | Second- |
| | | (SEQ ID NO: 24) | strand |
| | | | synthesis |
| 25 | KV5 | GGGCCCAGGCGGCCGAGCTCGTGCTGACTCAGTCTCCAGAC | Second- |
| | | (SEQ ID NO: 25) | strand |
| | | | synthesis |
| 26 | KV6 | GGGCCCAGGCGGCCGAGCTCCAGATGACCCAGTCTCCATCTTC | Second- |
| | | (SEQ ID NO: 26) | strand |
| | | | synthesis |
| 27 | KV7 | GGGCCCAGGCGGCCGAGCTCCAGATGACCCAGTCTCCTTCCAC | Second- |
| | | (SEQ ID NO: 27) | strand |
| | | | synthesis |
| 28 | KV8 | GGGCCCAGGCGGCCGAGCTCTGGATGACCCAGTCTCCATCC | Second- |
| | | (SEQ ID NO: 28) | strand |
| | | | synthesis |
| 29 | KV9 | GGGCCCAGGCGGCCGAGCTCCAGATGACCCAGTCTCCATCTGC | Second- |
| | | (SEQ ID NO: 29) | strand |
| | | | synthesis |
| 30 | KV10 | GGGCCCAGGCGGCCGAGCTCGTGTTGACGCAGTCTCCAGG | Second- |
| | | (SEQ ID NO: 30) | strand |
| | | | synthesis |
| 31 | KV11 | GGGCCCAGGCGGCCGAGCTCGTAATGACACAGTCTCCACCCAC | Second- |
| | | (SEQ ID NO: 31) | strand |
| | | | synthesis |
| 32 | KV12 | GGGCCCAGGCGGCCGAGCTCACACTCACGCAGTCTCCAGC | Second- |
| | | (SEQ ID NO: 32) | strand |
| | | | synthesis |
| 33 | LV1 | GGGCCCAGGCGGCCGAGCTCGCCCTGACTCAGCCTGCC | Second- |
| | | (SEQ ID NO: 33) | strand |
| | | | synthesis |
| 34 | LV2 | GGGCCCAGGCGGCCGAGCTCGCCCTGACTCAGCCTCCC | Second- |
| | | (SEQ ID NO: 34) | strand |
| | | | synthesis |
| 35 | LV3 | GGGCCCAGGCGGCCGAGCTCGTGCTGACTCAGCCACCC | Second- |
| | | (SEQ ID NO: 35) | strand |
| | | | synthesis |
| 36 | LV4 | GGGCCCAGGCGGCCGAGCTCGAGCTGACACAGCCACCC | Second- |
| | | (SEQ ID NO: 36) | strand |
| | | | synthesis |
| 37 | LV5 | GGGCCCAGGCGGCCGAGCTCGTGCTGACTCAATCATCCTCTGC | Second- |
| | | (SEQ ID NO: 37) | strand |
| | | | synthesis |
| 38 | LV6 | GGGCCCAGGCGGCCGAGCTCGTGCTGACTCAGCCGTCTTC | Second- |
| | | (SEQ ID NO: 38) | strand |
| | | | synthesis |
| 39 | LV7 | GGGCCCAGGCGGCCGAGCTCGTGGTGACTCAGGAGCCCTC | Second- |
| | | (SEQ ID NO: 39) | strand |
| | | | synthesis |
| 40 | LV8 | GGGCCCAGGCGGCCGAGCTCGTGCTGACTCAGCCACCTTC | Second- |
| | | (SEQ ID NO: 40) | strand |
| | | | synthesis |
| 41 | LV9 | GGGCCCAGGCGGCCGAGCTCGTCGTGACGCAGCCGCC | Second- |
| | | (SEQ ID NO: 41) | strand |
| | | | synthesis |
| 42 | LV10 | GGGCCCAGGCGGCCGAGCTCGCCCTGACTCAGCCTCGC | Second- |
| | | (SEQ ID NO: 42) | strand |
| | | | synthesis |
| 43 | LV11 | GGGCCCAGGCGGCCGAGCTCGAGCTGACTCAGCCACACTC | Second- |
| | | (SEQ ID NO: 43) | strand |
| | | | synthesis |
| 44 | LV12 | GGGCCCAGGCGGCCGAGCTCGTGCTGACTCAGCCAACCTC | Second- |
| | | (SEQ ID NO: 44) | strand |
| | | | synthesis |
| 45 | LV13 | GGGCCCAGGCGGCCGAGCTCGTGGTGACCCAGGAGCCATC | Second- |
| | | (SEQ ID NO: 45) | strand |
| | | | synthesis |
| 46 | LV14 | GGGCCCAGGCGGCCGAGCTCGAGCTGACTCAGGACCCTGC | Second- |
| | | (SEQ ID NO: 46) | strand |
| | | | synthesis |
| 47 | LV15 | GGGCCCAGGCGGCCGAGCTCGAGCTGATGCAGCCACCC | Second- |
| | | (SEQ ID NO: 47) | strand |
| | | | synthesis |
| 48 | LV16 | GGGCCCAGGCGGCCGAGCTCGAGCTGACACAGCCATCCTC | Second- |
| | | (SEQ ID NO: 48) | strand |
| | | | synthesis |
| 49 | LV17 | GGGCCCAGGCGGCCGAGCTCGTGCTGACTCAGCCCCCG | Second- |
| | | (SEQ ID NO: 49) | strand |
| | | | synthesis |
| 50 | KJ1 | GGAAGATCTAGAGGAACCACCTTTGATCTCCAGCTTGGTCCC | Second- |
| | | (SEQ ID NO: 50) | strand |
| | | | synthesis |
| 51 | KJ2 | GGAAGATCTAGAGGAACCACCTTTGATTTCCACCTTGGTCCCTTG | Second- |
| | | (SEQ ID NO: 51) | strand |
| | | | synthesis |
| 52 | KJ3 | GGAAGATCTAGAGGAACCACCTTTGATATCCACTTTGGTCCCAGG | Second- |
| | | (SEQ ID NO: 52) | strand |
| | | | synthesis |
| 53 | KJ4 | GGAAGATCTAGAGGAACCACCTTTAATCTCCAGTCGTGTCCCTTG | Second- |
| | | (SEQ ID NO: 53) | strand |
| | | | synthesis |
| 54 | LJ1 | GGAAGATCTAGAGGAACCACCGCCTAGGACGGTCAGCTTGGTCC | Second- |
| | | (SEQ ID NO: 54) | strand |
| | | | synthesis |
| 55 | LJ2 | GGAAGATCTAGAGGAACCACCGCCTAGGACGGTGACCTTGGTCC | Second- |
| | | (SEQ ID NO: 55) | strand |
| | | | synthesis |
| 56 | LJ3 | GGAAGATCTAGAGGAACCACCGCCGAGGACGGTCACCTTGGTG | Second- |
| | | (SEQ ID NO: 56) | strand |
| | | | synthesis |
| 57 | LJ4 | GGAAGATCTAGAGGAACCACCGCCGAGGGCGGTCAGCTGGG | Second- |
| | | (SEQ ID NO: 57) | strand |
| | | | synthesis |
| 58 | AMP_H_ | GGTGGTTCCTCTAGATCTTCCTCCTCTGGTGGCGGTGGCTCGGGCGGTGGTGGG | First |
| | forward | (SEQ ID NO: 58) | round |
| | | | PCR |
| 59 | AMP_H_ | CCTGGCCGGCCTGGCCAC (SEQ ID NO: 59) | First |
| | reverse | | round |
| | | | PCR |
| 60 | AMP_K/L_ | GGGCCCAGGCGGCCGAG (SEQ ID NO: 60) | First |
| | forward | | round |
| | | | PCR |
| 61 | AMP_K/L_ | GGAAGATCTAGAGGAACCACC (SEQ ID NO: 61) | First |
| | reverse | | round |
| | | | PCR |
| 62 | EXT-fwd | GGCGGGGCCCAGGCGGCCGAGCTC (SEQ ID NO: 62) | Overlap |
| | | | extension |
| | | | PCR |
| 63 | EXT-rev | GAGCCTGGCCGGCCTGGCCACTAGT (SEQ ID NO: 63) | Overlap |
| | | | extension |
| | | | PCR |

Then, the raw NGS forward (R1) and reverse (R2) reads were merged, filtered based on Phred scores, and subclustered using UMI using paired-end read merger (PEAR), v0.9.10, and Clustal Omega, v1.2.4, with the default setting[18, 19]. The sequence annotation process used the IMGT (International Immunogenetics Information System) constant gene database[20] to obtain the isotype, and IgBLAST, v1.17.1 to obtain VJ genes, CDR1/2/3 sequences, and the number of mutations from the corresponding V genes as previously described[4]. To eliminate the possibility of contamination due to index swapping within Illumina flow cells between samples, reads with identical sequences including the UMI from different samples were excluded on the basis that such an event is highly unlikely without contamination[22]. The combined NGS throughput of each sample is listed in Tables 1 and 2. Sequencing reads that have survived through the entire process is considered to be a productive BCR sequence.

### Example 3—BCR Repertoire Sharing and Exclusion Analysis

First, shared BCR clonotypes are defined in this study as a unique combination of V gene, J gene, and CDR3 amino acid sequence which are present in 2 or more subjects within a cohort. The shared BCR sequences in this context refer to the collective BCR sequences that can be grouped into each shared BCR clonotype.

Clonotypes which consist only of naïve sequences in the NGS data obtained the above Example 2, those with the IgM or IgD isotype with 1 or less somatic mutations were excluded from further analysis to account only for shared clonotypes arising from shared antigenic stimuli[3]. Due to potential risk of molecular contamination during the NGS library preparation stage, an additional strict in silico filtering step was employed. Shared clonotypes that have patient specific sets of unique BCR nucleotide sequences that are completely eclipsed by another patient's set was considered as contamination due to the low likelihood of 2 different people sharing only the same exact somatic mutations in the nucleotide level.

The similarity score based on shared cohort specific BCR clonotypes was calculated as the cosine similarity of the two vectors each representing a subject's repertoire. The vectorization process involved utilizing one-hot encoding to create a vector with the length equal to the total number of cohort specific and shared BCR clonotypes.

The presence or absence of each clonotypes for the patient was encoded as either the value 1 or 0. The cosine similarity being calculated as the inner product divided by the product of the size of each vector can be interpreted as the number of shared cohort specific shared clonotypes both present in a patient pair divided by the square root of the product of the total number of cohort specific shared clonotypes for each patient.

### Example 4—Construction of scFv Phage Display Library and Bio-Panning

In BCR repertoire of the above Example 3, for the V_{H} gene, V3-7/J1 VRLAEYFQN clonotype which is the most shared among all patients was chosen to test the affinity to Aβ42 peptide. In total, 16 sequences are included in that of clonotype and 9 out of 16 were presented in TP2. And 4 out of 9 sequences were synthesized in gBlocks Gene Fragments (Integrated DNA Technologies) depending on their sequence diversity of the CDR1, 2 regions at the amino acid level and somatic hypermutation status. For the construction of V_{κ}/V_{λ} shuffled libraries, total RNA from patients who possess the V3-7/J1 VRLAEYFQN clonotype in TP2 (AD9, AD14 and AD34) was used to synthesize cDNA using SuperScript IV First-Strand Synthesis System (Invitrogen) with gene-specific primers amplifying the J_{H} and C_{κ/λ} genes (No. 17, 18-20), respectively. Patient 35 also had that of heavy chain, however, experiments were preceded before he joined the second blood drawing. It was purified with SPRI beads (Beckman Coulter AMPure XP Reagents) at a 1:1.8 ratio and the purified cDNA was used for the first round of PCR synthesis using V_{κ}/V_{λ} and J_{κ}/J_{λ} gene-specific primers (No. 21-57; 95° C. for 3 min. 4 cycles of 98° C. for 1 min, 60° C. for 1 min, 72° C. for 1 min, and 72° C. for 10 min). Likewise, it was purified with SPRI beads at a 1:1 ratio. After that, both 4 synthesized V_{H} gene and V_{κ}/V_{λ} genes from 3 patients' total RNA were amplified using a KAPA Biosystems kit (Roche) with following primers (No. 58-61; 95° C. for 3 min. 27 cycles of 98° C. for 30 s, 65° C. for 30 s, 72° C. for 1 min, and 72° C. for 10 min). Then, each gene fragment of V_{H} and V_{κ}/V_{λ} was subjected to electrophoresis on a 1.5% agarose gel and purified through the size of each DNA using QIAquick gel extraction kit (QIAGEN) following the manufacturer's instructions. The amplified 4 V_{H} and V_{κ}/V_{λ} gene fragments from 3 patients were mixed at equal ratio to make a single pool of V_{H} and V_{κ}/V_{λ} gene, respectively. From the equally mixing pool of V_{H} and V_{κ}/V_{λ}, same amount (100 ng) of each gene fragments were subjected to overlap extension PCR to generate final scFv genes by using a KAPA Biosystems kit (Roche) with overlap extension primers (No. 62, 63:95° C. for 3 min. 25 cycles of 98° C. for 30 s, 65° C. for 30 s, 72° C. for 1 min 30 s, and 72° C. for 10 min). After that, the amplified scFv genes were purified and cloned into a phagemid vector as described previously[23].

Phage display of the human scFv library with 1.1×10⁹ colony-forming units was generated using 4 types of synthesized V_{H} gene fragment and shuffled V_{κ}/V_{λ} gene which derived from the cDNA of patient No. 6, 14 and 34. Following library was subjected to four rounds of bio-panning against the human Aβ42 peptide (Tocris Bioscience Amyloid β-Peptide 1-42 human, 1428) as described previously[24, 25]. Phage-display experiments including reamplification, rescue and PEG precipitation of library were mainly carried out with the protocols described in Phage Display: A Laboratory Manual [26]. Briefly, 2 μg of the Aβ42 peptide was coated to the surface of the immunotube with high binding surface (SPL Life Sciences, 43015) at 4° C. for 16 hours and blocked with 5 ml of 3% bovine serum albumin (BSA) in PBS (w/v). Then, the scFv phage library (about 1013 phages) was incubated for 2 hours at room temperature. During the first round of panning, the tube was washed once with 5 ml of 0.05% (v/v) Tween 20 (Sigma-Aldrich, P1379) in phosphate-buffered saline (PBST). After each round of panning, remaining phages bound to the coated antigen were eluted and rescued for the next round of bio-panning as described above. For the other rounds of panning, the number of washes was increased to three. To select antigen reactive scFv phage clones, each phage clones from the titration plate of the last round of panning was subjected to phage ELISA, using the antigen-coated microtiter plates, as described previously[26]. Reactive scFv clones were analyzed through Sanger nucleotide sequencing using their phagemid DNA extracted from the individual phage clones as discussed before[4]. A recombinant scFv protein tagged with human IgG1 FC (hFc) was expressed in mammalian expression system and purified as describe previously[4].

### Example 5—Enzyme-Linked Immunosorbent Assay

The specificity and antigen affinity of individual phage scFv clones and recombinant scFv-hFc fusion proteins of the above Example 4 were assessed by ELISA. 100 nM of human Aβ42 peptides, dissolved in 50 μl of 0.1 M sodium bicarbonate coating buffer (pH 8.6), were added to each well of 96-well microtiter plates (Corning, 3690) as described earlier. Shortly, the plates were coated with the antigen by incubation at 4° C. for 16 hours and blocked with 3% BSA in PBS (w/v) at 37° C. for 1 hours. After shaking off the blocking solution, serially diluted phage supernatant (twofold) or scfv-hFc (fivefold, S dilutions, starting from 1 μM to 12.8 pM) in blocking buffer were added to the wells of microtiter plates and incubated at 37° C. for 2 hours. Then, plates were washed three times with 0.05% PBST (v/v). For ELISAs with phage clones, HRP-conjugated rabbit antihuman IgG Fc antibody (Invitrogen, 31423, 1:5,000) and HRP-conjugated anti-M13 antibody (Sino Biological, 11973-MM05T-H, 1:4,000) were diluted in blocking buffer as manufacturer's protocols, followed by adding to wells and incubated at 37° C. for 1 hours. It performed respectively in the duplicated batch to determine the amount of antigen-reactive antibody or M13 bacteriophage. In ELISA for the recombinant scFvhFc fusion proteins, HRP-conjugated rabbit anti-human IgG Fc antibody (Invitrogen, 31423, 1:5,000) was added to wells and incubated at 37° C. for 1 hours. After washing three times with 0.05% PBST, 2,2′-azino-bis-3-ethylbenzothiazoline-6-sulfonic acid solution (Invitrogen, 002024) was added to the wells as an HRP substrate. Absorbance was measured at 415 nm, respectively, using a microplate spectrophotometer (Thermo Fisher Scientific Inc., Multiskan GO). All assays were carried out in duplicate.

### Example 6—Selection of Stereotypic AD Patient-Specific Non-Naïve BCR Clonotypes

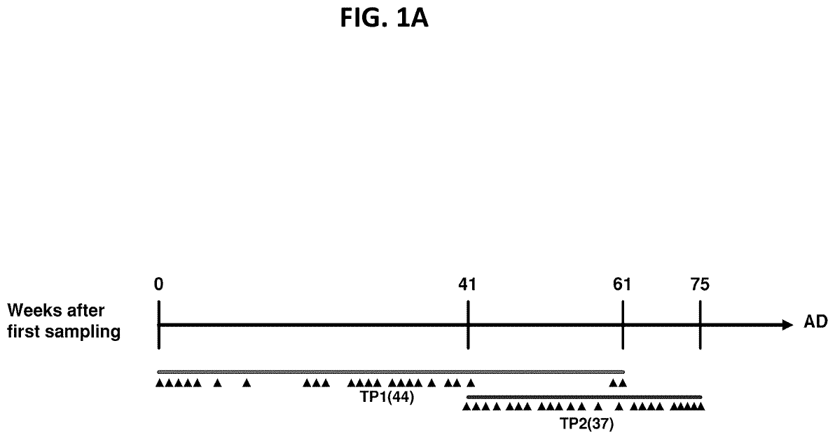

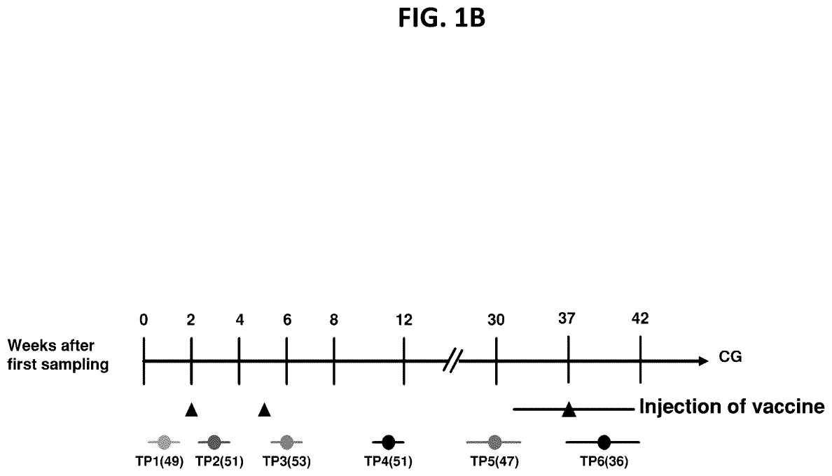

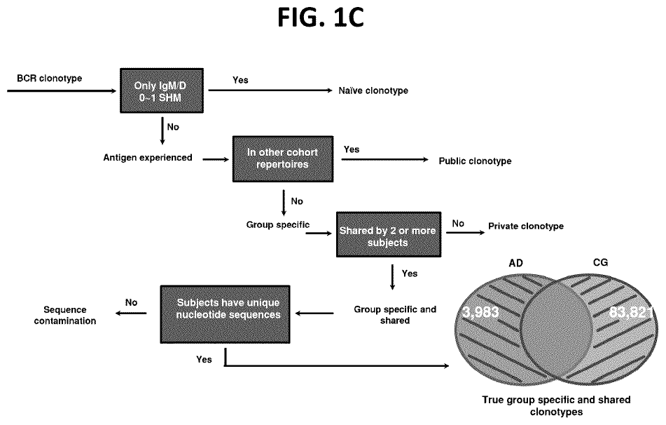

For a period of 75 weeks, peripheral blood (PB) samples were collected from 44 Alzheimer patients with a median age of 75 years (standard deviation; 7.04) and sex ratio of 34% and 66% (male vs female). Except for seven patients (Patient ID 3, 5, 8, 19, 28, 30, and 37), PB samples were collected twice with interval between 14 and 53 weeks (FIG. 1A). The duration for the first and second PB sampling was 61 and 34 weeks, respectively. From these 81 PB samples, cDNA was prepared and subjected to the amplification of a gene fragment encoding variable domain (VH) and Nterminal part of constant domain 1 (CH1) of B cell receptor (BCR) heavy chain using a specific primer set (Tables 1 and 2), which were analyzed in next generation sequencing with a median read of 245,033 (standard deviation of 184,462, Tables 4 and 5). For the analysis of BCR repertoire, we first removed naïve BCR sequences, which we define as those with IgM or ID isotype containing less than 1 somatic hypermutation (SHM). Then we grouped the remaining sequences into clonotypes, which was defined as a collection of sequences sharing the same CDR3 sequence at the amino acid level as well as encoded by identical IGHV and IGHJ genes[3]. Among these BCR clonotypes, AD patient-specific clonotypes were selected by excluding clonotypes found in a control group (CG) established in our prior studies, which was constituted with 55 vaccinees (285 PB samples obtained chronologically) injected three times with COVID-19 vaccine15 (FIG. 1B, Table 3). Control group specific non-naïve BCR clonotypes were also obtained following the same procedures. Before further analysis, we tried to remove any possibility of aerosol molecular contamination during NGS library preparation and index switching during sequencing[1]. For this, we excluded all the stereotypic clonotypes in which each patient or control did not show unique BCR sequences at the nucleotide level.

After all these selection processes we could select 3.983 and 83,821 BCR clonotypes specific to AD patient group or control group, respectively (FIG. 1C).

TABLE 3
Demographics of AD and CG group

| Characteristics | AD cohort | CG cohort |
|---|---|---|
| Number | 44 | 55 |
| Age (years) | 74.9 ± 6.9* | 33.2 ± 9.1 |
| Sex (M:F) | 15:29 | 26:29 |

AD, Alzheimers disease; approved under the recommendations of the Institutional Review Board (IRB) of the Seoul National University Bundang Hospital (SNUBH, IRB approval number, B-2102-664-305), South Korea.
CG, Vaccination control group; approved by the IRB of Seoul National University Hospital (SNUH, IRB approval number, 2102-032-1193), South Korea.
*Age based on time of first sampling (TP1)

TABLE 4
Vaccine cohort

| subject | TP1 | TP2 | TP3 | TP4 | TP5 | TP6 |
|---|---|---|---|---|---|---|
| 1 | 116846 | 74116 | N/A | 308607 | 503110 | 363352 |
| 2 | N/A | 412242 | 359933 | 637596 | N/A | N/A |
| 3 | N/A | 233382 | 386838 | 207509 | 200078 | 392932 |
| 4 | 297560 | 295471 | 288481 | 298874 | 225565 | 159801 |
| 5 | 277423 | 338856 | 276439 | 292434 | 100454 | 328502 |
| 6 | 117645 | 304697 | 281448 | 261449 | N/A | 90170 |
| 7 | 167730 | 447446 | 396370 | 425845 | N/A | 308297 |
| 8 | 201381 | 272153 | 238318 | 250530 | 98330 | N/A |
| 9 | 71061 | 312156 | 268349 | 146753 | 177497 | 166913 |
| 10 | 110138 | 328043 | 321325 | 225229 | 311158 | 426625 |
| 11 | 115745 | 304497 | 349801 | 219420 | 296670 | N/A |
| 12 | 83882 | 367630 | 386806 | 480477 | N/A | N/A |
| 13 | 94375 | 285776 | 365978 | 210716 | 540447 | 132993 |
| 14 | 136815 | 277598 | 73364 | 394457 | 452999 | 398426 |
| 15 | 257019 | 287627 | 352889 | 436163 | 292988 | 131853 |
| 16 | 69313 | 264901 | N/A | N/A | 380708 | N/A |
| 17 | 186003 | 254969 | 411110 | 155924 | 185741 | 433726 |
| 18 | 143543 | 89465 | 447991 | 352477 | 384304 | N/A |
| 19 | 188195 | N/A | 434379 | 411592 | 535408 | N/A |
| 20 | 79075 | 763067 | 332463 | 634987 | N/A | N/A |
| 21 | 130318 | 44297 | 251913 | 327060 | 236588 | 526006 |
| 22 | 108257 | 127565 | 611092 | 186488 | N/A | 239071 |
| 23 | 106012 | N/A | 622746 | 193718 | 343870 | 182295 |
| 24 | 86189 | 125168 | 381850 | N/A | 135668 | N/A |
| 25 | 217788 | 290448 | 307991 | 244198 | 386140 | N/A |
| 26 | 268423 | 269479 | 287259 | 527800 | 115860 | N/A |
| 27 | 276442 | 294510 | 397970 | 327793 | 470213 | 494132 |
| 28 | 73922 | 170608 | 229818 | 153453 | 298818 | 272185 |
| 29 | 106076 | 160933 | 284978 | 198595 | 210089 | N/A |
| 30 | 350181 | 137372 | 376001 | 93516 | 98233 | 172479 |
| 31 | 57928 | 156320 | 712424 | 209826 | 217594 | N/A |
| 32 | 520050 | 186185 | 506773 | 289453 | 353165 | 543473 |
| 33 | 139592 | 161044 | 272372 | 196285 | 327832 | 297902 |
| 34 | 111086 | 250341 | 283938 | 194308 | 59673 | N/A |
| 35 | 164995 | 234015 | 416674 | N/A | 371122 | 354917 |
| 36 | 108078 | 306270 | 266945 | 137483 | 274328 | 289645 |
| 37 | N/A | 915107 | 335969 | 391259 | 409694 | N/A |
| 38 | 205087 | 357783 | 379389 | 221707 | 668335 | 379577 |
| 39 | 180835 | 355525 | 121246 | N/A | 255377 | 208146 |
| 40 | 151495 | 223316 | 133059 | 178507 | 322811 | 112402 |
| 41 | 171548 | N/A | 389549 | 186037 | 243664 | 132788 |
| 42 | 135524 | 271827 | 295086 | 239576 | N/A | N/A |
| 43 | 166742 | 239590 | 128758 | 64537 | 123037 | 301277 |
| 44 | 137807 | 367324 | 494786 | 91489 | 661630 | 363467 |
| 45 | 92714 | 215559 | 523444 | 94863 | 253647 | 131049 |
| 46 | N/A | 301442 | 597236 | 219932 | N/A | N/A |
| 47 | 69303 | 279692 | 447821 | 116292 | 184017 | N/A |
| 48 | N/A | 274990 | 264335 | 277660 | 339829 | 103204 |
| 49 | 170408 | 253677 | 312839 | 146165 | 175187 | 435089 |
| 50 | 235553 | 310495 | 234564 | 153694 | 383540 | 395902 |
| 51 | 19505 | N/A | 290752 | 125531 | 280175 | N/A |
| 52 | 86072 | 389170 | 441317 | 201485 | 373482 | 276724 |
| 53 | 232760 | 345513 | 541998 | 109294 | 259369 | 302281 |
| 54 | 66017 | 300447 | 565089 | 125593 | 176918 | 92764 |
| 55 | N/A | 314304 | 276673 | 79930 | 277668 | 375069 |

TABLE 5
Alzheimer's disease

| subject | TP1 | TP2 |
|---|---|---|
| 1 | 278490 | 298543 |
| 2 | 482372 | 386775 |
| 3 | 375502 | N/A |
| 4 | 238549 | 255707 |
| 5 | 430371 | N/A |
| 6 | 216502 | 172377 |
| 7 | 163212 | 228539 |
| 8 | 357746 | N/A |
| 9 | 462337 | 264165 |
| 10 | 736856 | 190256 |
| 11 | 646467 | 376727 |
| 12 | 134150 | 177258 |
| 13 | 644200 | 126737 |
| 14 | 403475 | 334311 |
| 15 | 592160 | 139445 |
| 16 | 333471 | 367383 |
| 17 | 230042 | 235500 |
| 18 | 291166 | 301188 |
| 19 | 639201 | N/A |
| 20 | 168072 | 132959 |
| 21 | 193563 | 223286 |
| 22 | 263507 | 280624 |
| 23 | 252792 | 56821 |
| 24 | 383144 | 171485 |
| 25 | 457475 | 161323 |
| 26 | 82630 | 135646 |
| 27 | 138279 | 138835 |
| 28 | 116251 | N/A |
| 29 | 454885 | 245033 |
| 30 | 851831 | N/A |
| 31 | 317011 | 122818 |
| 32 | 506812 | 286435 |
| 33 | 724754 | 108953 |
| 34 | 919858 | 193478 |
| 35 | 225313 | 164490 |
| 36 | 235339 | 112636 |
| 37 | 658256 | N/A |
| 38 | 392077 | 205251 |
| 39 | 194282 | 178175 |
| 40 | 267038 | 177530 |
| 41 | 38294 | 63111 |
| 42 | 141594 | 172079 |
| 43 | 262308 | 192285 |
| 44 | 273453 | 331364 |

### Example 7 Stereotypic AD Patient-Specific Non-Naïve Clonotypes Showed Significantly Higher Degree of Overlap in Patient Pairs Compared to CG Pairs

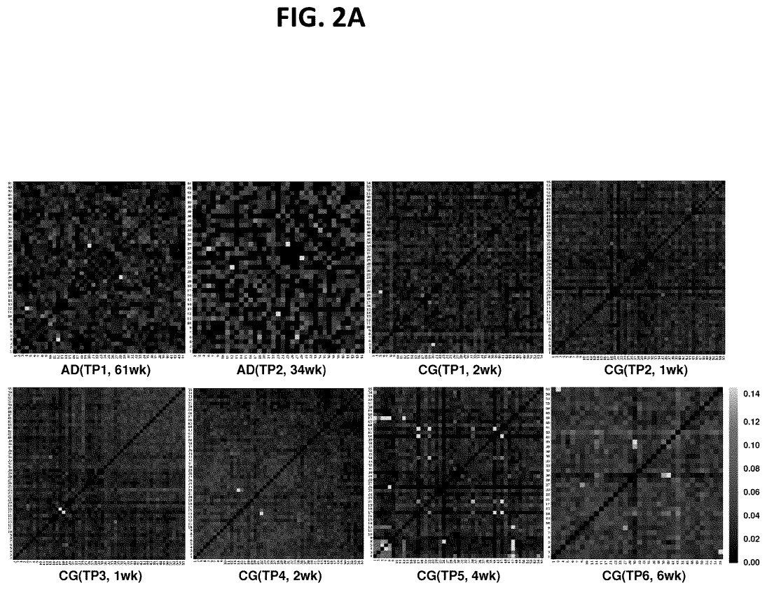

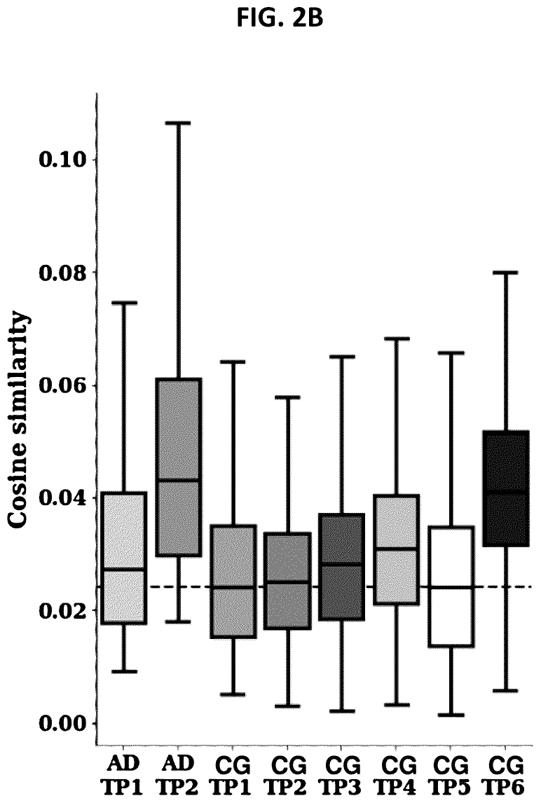

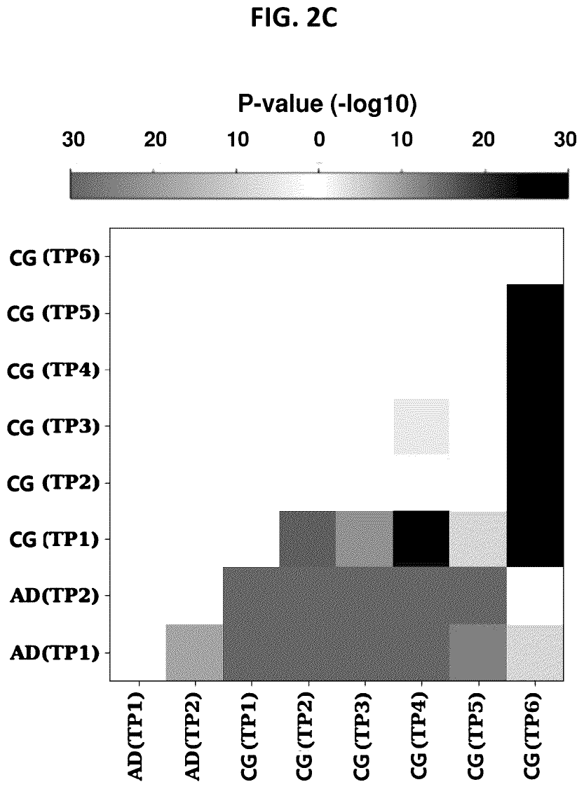

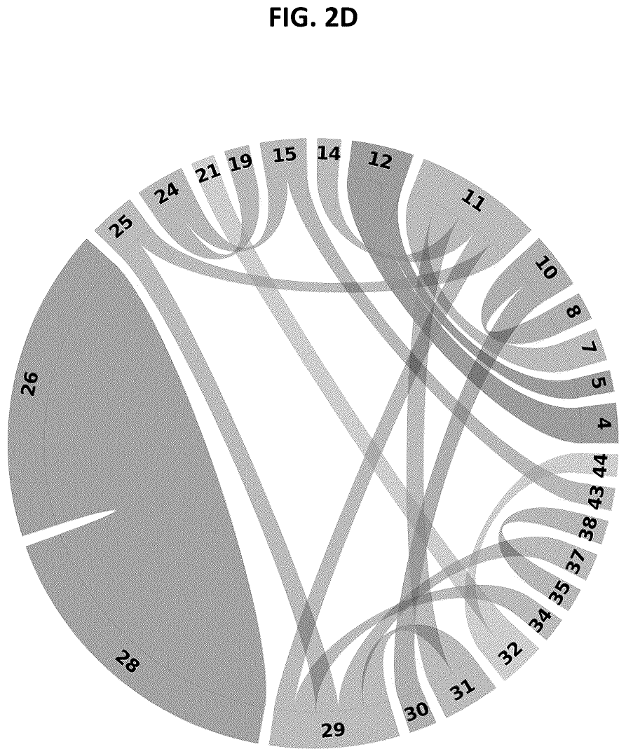

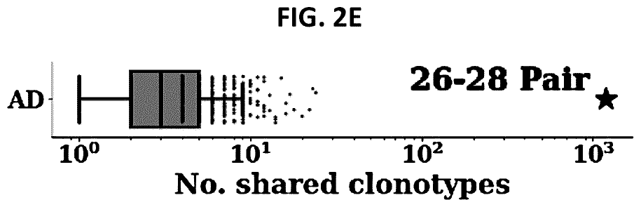

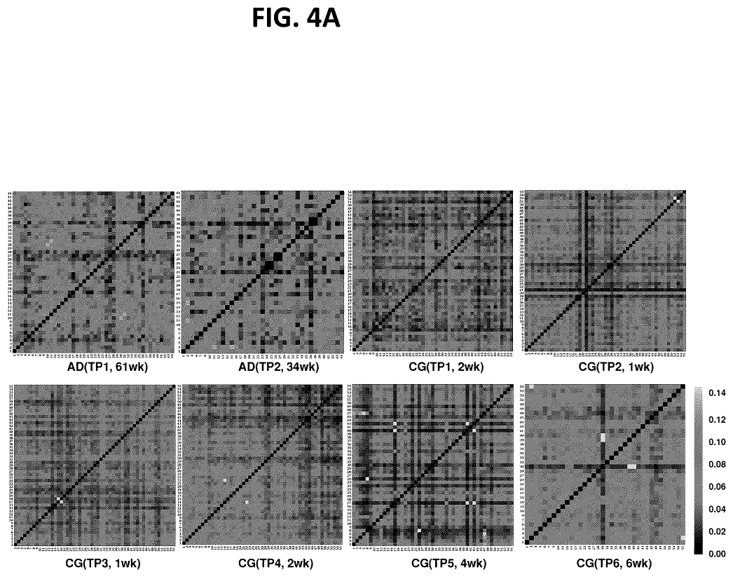

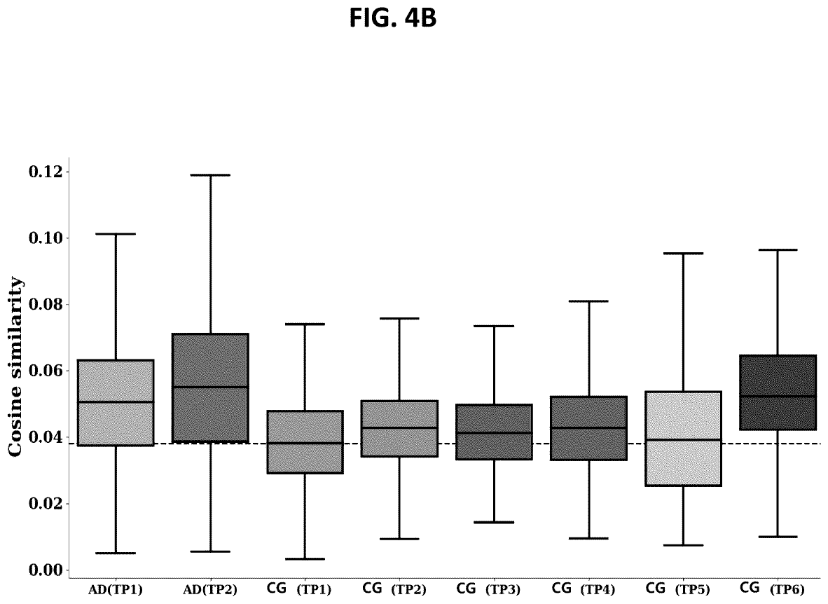

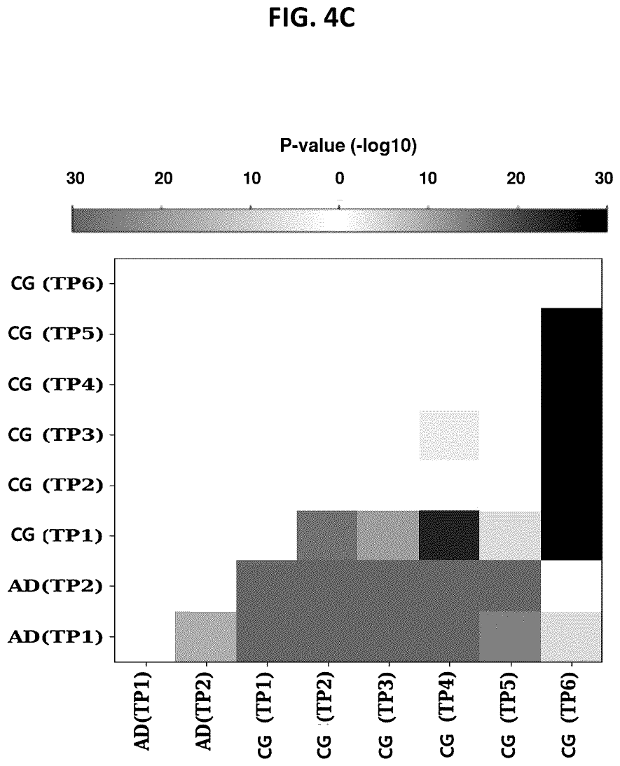

Then, the present inventors analyzed the distribution of stereotypic AD patient-specific BCR clonotypes in 1,892 ((44 X 44)-44) or 1,332 ((37 X 37)-37) patient pairs in the first and the second PB samples, respectively. The present inventors calculated the degree of overlap for stereotypic BCR clonotypes in each pair as the cosine similarity of one-hot encoded vectors representing the presence or absence of each stereotypic clonotype in a patient's BCR repertoire. The present inventors also obtained the degree of overlap for the CG pairs in six PB sampling points. As expected, there was dramatic increase in the average cosine similarity at time point (TP) 3, the TP4 and TP6 PB samples obtained right after the second-, 1 month after the second-, and right after the third-injection of COVID-19 vaccine, respectively, due to the concerted BCR response toward the immunogen compared to the pre-vaccinated state (TP1) (FIG. 2A-C, p=4.92×10⁶, p=2.52×10⁻³⁰, p=2.26×10⁻¹¹²). AD patient pairs showed much higher average cosine similarity at both sampling points compared to CG TP1 with statistical significance (p=2.06×10⁻²¹, p=1.22×10⁻¹⁶⁹). The value was higher at the second PB samples and might be originated from shorter PB sampling duration (61 weeks vs 34 weeks), which would provide less diverse seasonal antigens to AD patients. It was quite astonishing that the average cosine similarity of the second PB samples was higher than that of TP6 PB samples of CG showing the highest value among CG PB samples with statistical significance (p=1.86×10⁻⁶). This observation was extra-ordinary when we consider that PB sampling duration was much longer in AD patients (34 weeks vs 6 weeks). As the present inventors do not aware of COVID-19 infection history or vaccination of AD patients, the present inventors performed the same analysis without excluding stereotypic BCR clonotype of AD patients from stereotypic clonotypes of CG. The cosine similarity value of AD patients' PB samples at both TP1 and TP2 were higher than those of CG's PB samples with statistical significance at all TPs except for TP6, which were similar (FIGS. 4A-C). These analysis clearly showed that AD patients shares stereotypic BCR clonotypes at an equivalent or higher degree compatible to vaccinees who received their third injection for the previous six weeks before PB sampling irrespective of AD patients much longer PB sampling period of 34 weeks.

Analysis of the present inventors showed that the degree of overlap on the stereotypic AD patient-specific BCR clonotypes significantly differs in patients pairs suggesting the existence of patient subgroups categorized by their BCR clonotypes. To find these patient subgroups, the stereotypic AD patient-specific BCR clonotypes at all TPs in each patient were combined and the cosine similarity of patient pairs were calculated. Twenty patient pairs with the highest cosine value were selected and displayed in Chord plot (FIG. 2D), in which the patients were divided to six subgroups. Among these pairs, one patient pair (patient ID 26 and 28) had an outlying degree of cosine similarity value of 0.961. And the number of BCR clonotypes was also extraordinarily high (FIG. 2E). Indeed, among 1,225 and 1,228 stereotypic BCR clonotypes in patient ID 26 and patient ID 28, respectively. 1,179 clonotypes were shared by these two patients. The presence of AD patient subgroups showing high degree of overlap in their BCR clonotypes strongly suggested the existence of common and possibly auto-antigens shared by the subgroup.

Results of the present inventors suggest that the antibodies encoded by stereotypic AD patient-specific BCR clonotypes are developed by chronic exposure to autoantigens through the progression of disease.

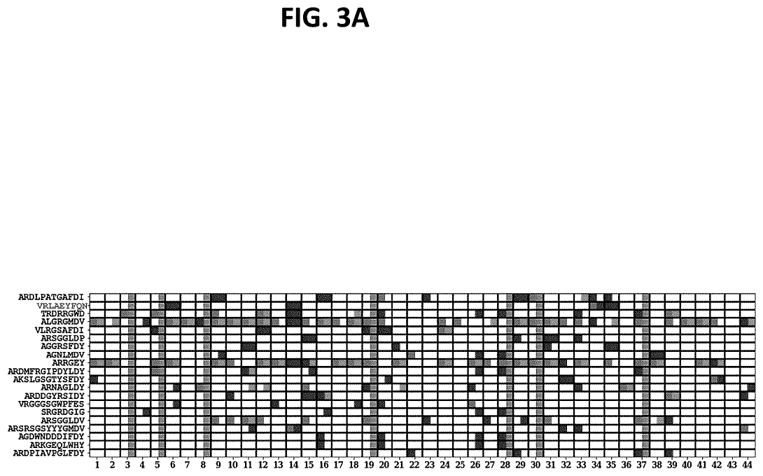

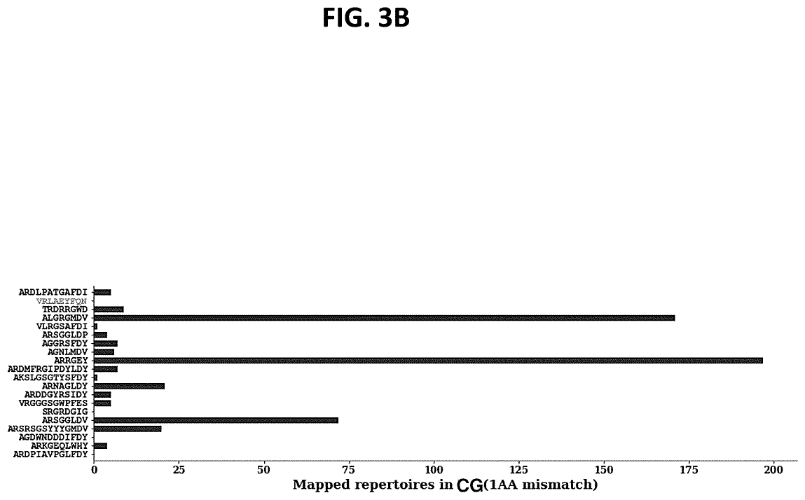

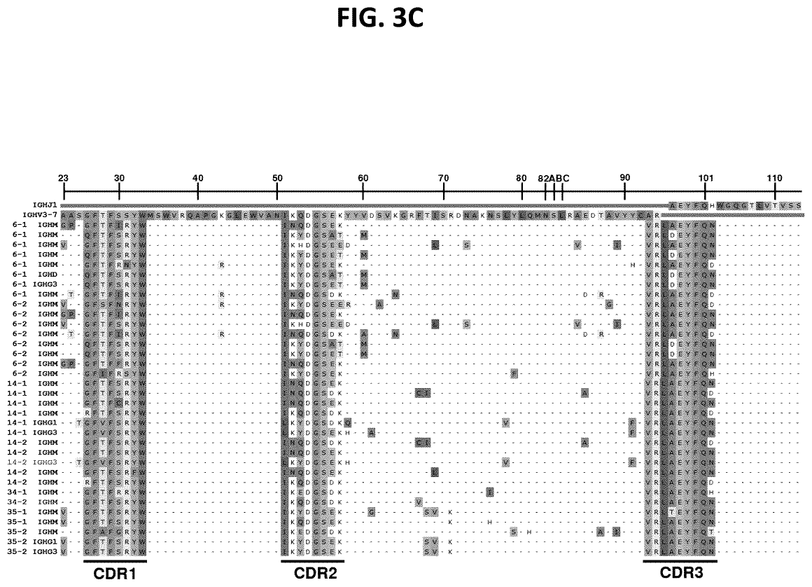

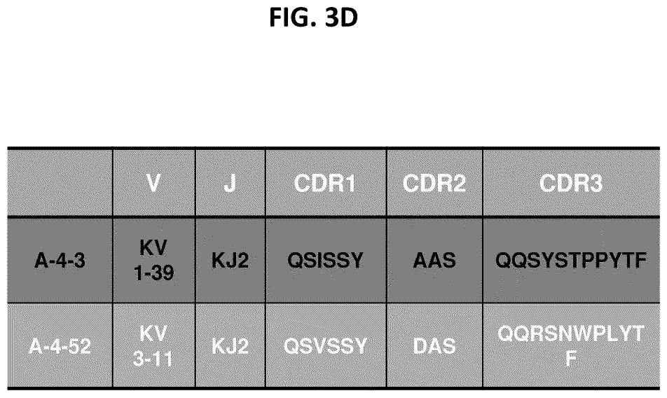

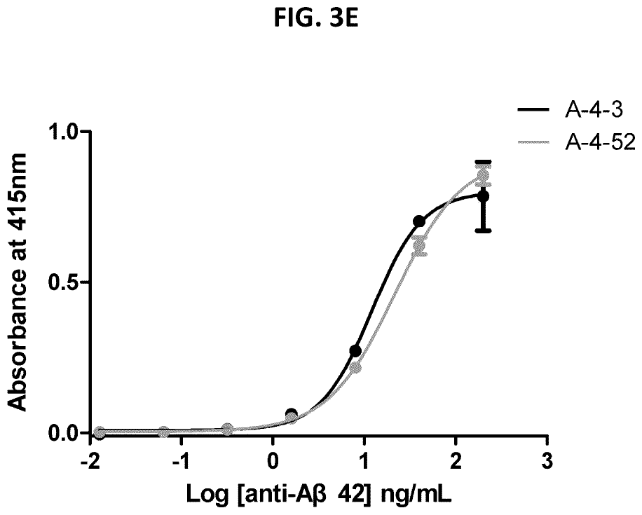

It can be confirmed that the presence of common immunogen among AD patients shaping their BCR repertoire to coordinated directions through this. It was also found that many of these stereotypic AD patient-specific BCR clonotypes or their very homologous clonotypes persisted over a period of 14 and 53 weeks (FIG. 3A). And when examining one group of long-lasting homologous BCR clonotypes at the individual sequence level, the present inventors could find not only the occurrence of CSR but also the accumulation of SHM at degree achievable only after the third injection of COVID-19 vaccine[1] (FIG. 3B). This finding revealed that the exposure to this common immunogen was not a single event in AD patients sharing these homologous clonotypes.

Collectively, the above results of the present inventors strongly suggested the existence of common autoantigens persisting inside AD patients provoking concerted humoral immunity.

### Example 8—a Highly Shared and Persistent AD Patient-Specific BCR Clonotype Encoded Antibody Reactive to Aβ42 Peptide

To identify the antigens to which these stereotypic AD patient-specific BCR clonotypes are reactive, we selected twenty most highly shared clonotypes present in four or higher number of patients (FIG. 3A). We also focused on the persistence of these clonotypes through the two TPs. One clonotype encoded by IGHV3-7/IGHJ1 genes with the CDR3 sequence of VRLAEYFQN were present at both TPs in three patients. And when we allow one amino acid mismatch in CDR3, one more patient (patient ID 34) with the similar clonotype (CDR3 sequence: VRLAEYFQH) at TP1 was found (FIG. 3A). In 285 PB samples of CG, we could not find any BCR clonotypes encoded by IGHV3-7/IGHJ1 genes and with CDR3 sequences homologous to VRLAEYFQN with amino acid mismatch allowance (FIG. 3B). The present inventors collected BCR sequences of all clonotypes encoded by IGHV3-7/IGHJ1 and with CDR3 sequences homologous to VRLAEYFQN with one amino acid mismatch allowance (FIG. 3C). The class switch recombination (CSR) to IgG1 and IgG3 was found in three patients. And the BCR sequences harbored somatic hypermutation (SHM) ranging from 4 to 19 in CDR1, CDR2 and the framework regions. As the occurrence of CSR and accumulation of SHM on these BCR sequences is the hallmark of chronic repeated exposure to antigen[4], there was a possibility for these clonotypes to develop by autoantigen.

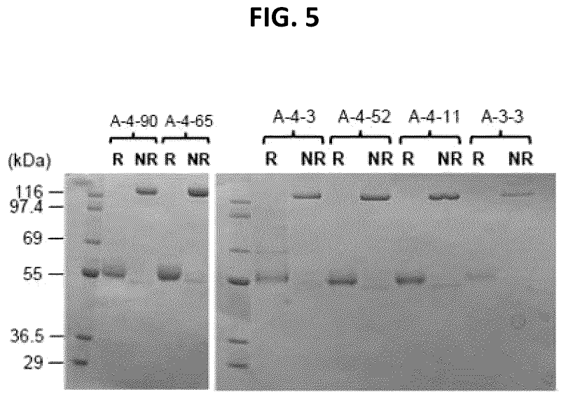

Among these BCRs, the present inventors synthesized four BCR heavy chain genes (Table 6) and used them to construct a single-chain variable fragment (scFv) phage display library using the amplified light chain genes of AD patients (patient ID 6, 14, and 34) possessing IGHV3-7/IGHJ1 VRLAEYFQN clonotype. Then the present inventors performed biopanning of this library on Aβ42 peptide, a well-known autoantigen in AD [2], and could get antigen-reactive scFv clones. When the present inventors expressed these clones as recombinant scFv-human Fc fusion proteins and performed an ELISA, two scFv clones with different k light chains (FIGS. 3C, and 5) showed reactivity against the Aβ42 peptide, with half-maximal effective concentrations (EC50) of 48.37 nM and 58.27 nM, respectively (FIG. 3D).

TABLE 6
Synthesized BCR heavy chain sequences

| Se- | Con- | | | | | | Somatic |
|---|---|---|---|---|---|---|---|
| quence | verged | | V | J | C | | hyper- |
| ID | patients | Germline_alignment | gene | gene | gene | Sequence | mutation |
| SNUBH- | SNUBH- | TGCAGCCTCTGGATTCACCTTTAGTAG | IGHV3- | IGHJ1 | IGHM | GAGGTGCAGCTGGTGGAGTCTGGGG | 5 |
| 34-2- | 14- | ATATTGGATGAGCTGGGTCCGCCAGG | 7*01 | *01 | *04 | GAGGCTTGGTCCAGCCTGGGGGGTC | |
| 91969 | 2\|SNUBH- | CTCCAGGGAAGGGGCTGGAGTGGGT | | | | CCTGAGACTCTCCTGTGCAGCCTCTG | |
| | 34- | GGCCAACATAAAGCAAGATGGAAGT | | | | GATTCACCTTTAGTAGATATTGGATGA | |
| | 2\|SNUBH- | GACAAATACTATGTGGACTCTGTGAA | | | | GCTGGGTCCGCCAGGCTCCAGGGAA | |
| | 6-2 | GGGCCGAGTCACCATCTCCAGAGACA | | | | GGGGCTGGAGTGGGTGGCCAACATA | |
| | | ACGCCAAGAACTCACTGTATCTGCAA | | | | AAGCAAGATGGAAGTGACAAATACTA | |
| | | ATGAACAGCCTGAGAGCCGAGGACA | | | | TGTGGACTCTGTGAAGGGCCGAGTCA | |
| | | CGGCTGTATATTACTGTGTGAG---- | | | | CCATCTCCAGAGACAACGCCAAGAAC | |
| | | GCTGAATACTTCCAGCACTGGGGCCA | | | | TCACTGTATCTGCAAATGAACAGCCTG | |
| | | GGGCACCCTGGTCACCGTCTCCTCA | | | | AGAGCCGAGGACACGGCTGTATATTA | |
| | | (SEQ ID NO: 64) | | | | CTGTGTGAGACTCGCTGAATACTTCCA | |
| | | | | | | GAACTGGGGCCAGGGCACCCTGGTC | |
| | | | | | | ACCGTCTCCTCA | |
| | | | | | | (SEQ ID NO: 65) | |
| SNUBH- | SNUBH- | TGCAGCCACTGGATTCGTCTTTAGTCG | IGHV3- | IGHJ1 | IGHG3 | GAGGTGCAGCTGGTGGAGTCTGGGG | 17 |
| 14-2- | 14- | TTATTGGATGAGTTGGGTCCGCCAGG | 7*03 | *01 | *29 | GAGGCTTGGTCCAGCCTGGGGGGTC | |
| 24582 | 2\|SNUBH- | CTCCAGGGAAGGGGCTGGAGTGGGT | | | | CCTGAGACTCTCCTGTGCAGCCACTG | |
| | 34- | GGCCAATTTAAAATATGATGGAAGTG | | | | GATTCGTCTTTAGTCGTTATTGGATGA | |
| | 2\|SNUBH- | AGAAACACTATGTGGACTCTGTGAAG | | | | GTTGGGTCCGCCAGGCTCCAGGGAA | |
| | 6-2 | GGCCGATTCACCATCTCCAGAGACAA | | | | GGGGCTGGAGTGGGTGGCCAATTTA | |
| | | CGCCAAGAACTCAGTCTATCTGCAAAT | | | | AAATATGATGGAAGTGAGAAACACTA | |
| | | GAACAGCCTGAGAGCCGAGGACACG | | | | TGTGGACTCTGTGAAGGGCCGATTCA | |
| | | GCCGTGTATTTCTGTGTAAG---- | | | | CCATCTCCAGAGACAACGCCAAGAAC | |
| | | GCTGAATACTTCCAGCACTGGGGCCA | | | | TCAGTCTATCTGCAAATGAACAGCCTG | |
| | | GGGCACCCTGGTCACCGTCTCCTCA | | | | AGAGCCGAGGACACGGCCGTGTATTT | |
| | | (SEQ ID NO: 66) | | | | CTGTGTAAGACTCGCTGAATACTTTCA | |
| | | | | | | GAACTGGGGCCAGGGCACCCTAGTC | |
| | | | | | | ACCGTCTCCTCA | |
| | | | | | | (SEQ ID NO: 67) | |
| SNUBH- | SNUBH- | TGCAGCCTCTGGATTCACCTTTAGTAG | IGHV3- | IGHJ1 | IGHM | GAGGTGCAGCTGGTGGAGTCTGGGG | 7 |
| 14-2- | 14- | GTTTTGGATGAGTTGGGTCCGCCAGG | 7*03 | *01 | *04 | GAGGCTTGGTCCAGCCTGGGGGGTC | |
| 24702 | 2\|SNUBH- | CTCCAGGGAAGGGGCTGGAGTGGGT | | | | CCTGAGACTCTCCTGTGCAGCCTCTG | |
| | 34- | GGCCAACATAAACCAAGATGGAAGTG | | | | GATTCACCTTTAGTAGGTTTTGGATGA | |
| | 2\|SNUBH- | AGAAATACTATGTGGACTCTGTGAAG | | | | GTTGGGTCCGCCAGGCTCCAGGGAA | |
| | 6-2 | GGCCGATTCACCCTCTCCAGAGACAA | | | | GGGGCTGGAGTGGGTGGCCAACATA | |
| | | CGCCAAGAACTCACTGTATTTGCAAAT | | | | AACCAAGATGGAAGTGAGAAATACTA | |
| | | GAACAGTCTGAGAGCCGAGGACACG | | | | TGTGGACTCTGTGAAGGGCCGATTCA | |
| | | GCCGTGTATTACTGT--------- | | | | CCCTCTCCAGAGACAACGCCAAGAAC | |
| | | GCTGAATACTTCCAGCACTGGGGCCA | | | | TCACTGTATTTGCAAATGAACAGTCTG | |
| | | GGGCACCCTGGTCACCGTCTCCTCA | | | | AGAGCCGAGGACACGGCCGTGTATTA | |
| | | (SEQ ID NO: 68) | | | | CTGTGTAAGGCTCGCTGAATACTTCCA | |
| | | | | | | GAACTGGGGCCAGGGCACCCTGGTC | |
| | | | | | | ACCGTCTCCTCA | |
| | | | | | | (SEQ ID NO: 69) | |
| SNUBH- | SNUBH- | TGTAGCCTCTGGATTCACCTTTAGTAG | IGHV3- | IGHJ1 | IGHM | GAGGTGCAGCTGGTGGAGTCTGGGG | 11 |
| 6-2- | 14- | GTATTGGATGAGCTGGGTCCGCCAGG | 7*01 | *01 | *04 | GAGGCTTGGTCCAGCCTGGGGGGTC | |
| 27474 | 2\|SNUBH- | CTCCAGGGAAGGGGCTGGAGTGGGT | | | | CCTGAGACTCTCCTGTGTAGCCTCTGG | |
| | 34- | GGCCAACATAAAGCACGATGGCAGTG | | | | ATTCACCTTTAGTAGGTATTGGATGAG | |
| | 2\|SNUBH- | AGGAAGACTATGTGGACTCTGTGAAG | | | | CTGGGTCCGCCAGGCTCCAGGGAAG | |
| | 6-2 | GGCCGATTCACCCTCTCCAGAGACAG | | | | GGGCTGGAGTGGGTGGCCAACATAA | |
| | | CGCCAAGAACTCACTGTATCTGCAAAT | | | | AGCACGATGGCAGTGAGGAAGACTAT | |
| | | GAACAGCCTGAGAGTCGAGGACACG | | | | GTGGACTCTGTGAAGGGCCGATTCAC | |
| | | GCTATATATTACTGTG-- | | | | CCTCTCCAGAGACAGCGCCAAGAACT | |
| | | CGTTTGGCTGAATACTTCCAGCACTG | | | | CACTGTATCTGCAAATGAACAGCCTGA | |
| | | GGGCCAGGGCACCCTGGTCACCGTCT | | | | GAGTCGAGGACACGGCTATATATTACT | |
| | | CCTCA (SEQ ID NO: 70) | | | | GTGTTCGTTTGGCTGAATACTTCCAGA | |
| | | | | | | ACTGGGGCCAGGGCACCCTGGTCAC | |
| | | | | | | CGTCTCCTCA (SEQ ID NO: 71) | |

With this, the present inventors identified BCR clonotypes that are AD specific and convergent among multiple patients using a total of 81 repertoires from 44 patients. Despite the stringent filtering process, both distribution and clonotype cosine similarity of stereotypic group specific clonotypes among Alzheimer patients were significantly increased compared to the repertoires of VC. When it comes to individual clonotypes, among the top 20 highly shared in AD patients, the V3-7/J1 VRLAEYFQN clonotype showed distinct characteristics such as full persistence in all possessing patients, exclusion from VC repertoires even for the extended clonotype, class switching in multiple patients, and a highly diverse mutation profile. Therefore, it was chosen for phage library construction and subsequent bio-panning. As a result, two scFv clones showed binding affinity against the human Aβ42 peptide.

Through this, it was confirmed that the composition according to one aspect of the present invention has an excellent effect in predicting or diagnosing Alzheimer's disease by simply using peripheral blood mononuclear cells isolated from a subject, in addition to methods such as MRI and PET scans using radioactive substances, by comprising a detecting agent of a B cell receptor (BCR) clonotype specific to an Alzheimer's disease patient.

### REFERENCES

1. Park, S., et al. An ancestral SARS-CoV-2 vaccine induces anti-Omicron variants antibodies by hypermutation. bioRxiv, 2023.2003.2015.S32728 (2023).
2. Kronimus, Y., et al. Fc N-glycosylation of autoreactive Aβ antibodies as a blood-based biomarker for Alzheimers disease. Alzheimers Demen (2023).
3. Ghraichy, M., et al. Maturation of the Human Immunoglobulin Heavy Chain Repertoire With Age. Frontiers in Immunology 11(2020).
4. Kim, S. I. et al. Stereotypic neutralizing V(H) antibodies against SARS-CoV-2 spike protein receptor binding domain in patients with COVID-19 and healthy individuals. Sci Transl Med 13(2021).
5. Laforce, R, Jr., et al. Molecular imaging in dementia: Past present and future. Alzheimers Dement 14, 1522-1552 (20181.
6. Chandra, A., et al. Applications of amyloid, tau, and neuroinflammation PET imaging to Alzheimers disease and mild cognitive impairment. Hum Brain Mapp 40, 5424-5442 (2019).
7. Jack, C. R., Jr, et al. Brain β-amyloid load approaches a plateau. Neurology 80, 890-896(2013).
8. Jagust W. J. & Landau, S. M. Temporal Dynamics of β-Amyloid Accumulation in Aging and Alzheimer Disease. Neurology 96, e1347-e1357 (2021).
9. Donohue, M. C., et al. Association Between Elevated Brain Amyloid and Subsequent Cognitive Decline Among Cognitively Normal Persons. Jama 317, 230S-2316 (2017).
10. Leal, S., Lockhart, S. N., Mas, A. Bell, R. K. & Jagust, W. J. Subthreshold Amyloid Predicts Tau Deposition in Aging. J Neurosci 38, 4482-4489 (2018).
11. Hansson, O., Blennow, K. Zetterberg H. & Dage J. Blood biomarkers for Alzheimer's disease in clinical practice and trials. Nature Aging 3, 506-519 (2023).
12. Ossenkoppele, A, van der Kant, R. & Hansson O. Tau biomarkers in Alzheimer's disease: towards implementation in clinical practice and trials. Lancet Neurol 21, 726-734 (2022).
13. Bernabeu-Zomoza, A., et al. Aβ42 Peptide Promotes Proliferation and Gliogenesis in Human Neural Stem Cells. Molecular Neurobiology 56, 4023-4036 (2019).
14. Wang, Q., et al. Design and Production of Bispecific Antibodies, Antibodies 8, 43 (2019).
15. Wu, J. & Li, L. Autoantibodies in Alzheimer's disease potential biomarkers, pathogenic roles, and therapeutic implications. J Biomed Res 30, 361-372 (2016).
16. Han, J. W. et al. Overview of the Korean Longitudinal Study on Cognitive Aging and Dementia. Psychistry Investig 15, 767-774 (2018).
17. Lee, H., et al. Optimization of peripheral blood volume for in silico reconstitution of the human B-cell receptor repertoire. FEBS Open Bio 12, 1634-1643 (2022)
18. Stevers, F. & Higgins, D. G. Clustal Omega for making accurate alignments of many protein sequences. Protein Sci 27, 135-145 (2018).
19. Zhang, J., Kobert, K., Flouri, T. & Stamatakis, A. PEAR: a fast and accurate illumina Paired-End reAd mergeR. Bioinformatics 30, 614-620 (2014).
20. Lefranc, M. P. IMGT databases, web resources and tools for immunoglobulin and T cell receptor sequence analysis, http//imgt.cinesfr. Leukemia 17, 260-266 (2003)
21. Ye, J., Ma, N., Madden, T. L. & Ostell, J. M. IgBLAST: an immunoglobin variable domain sequence analysts tool. Nucleic Acid Res 41, W34-40 (2013).
22. Griffiths, J. A. Richard, A. C., Bach, K., Lun, A. T. L. & Marioni, J. C. Detection and removal of barcode swapping in single-cell RNA-seq data. Nat Commun 9, 2667 (2018).
23. Andris-Widhopf, J. Steinberger, P., Fuller, R., Rader, C. & Barbas, C. F. 3rd. Generation of human scFv antibody libraries. PCR amplification and assembly of light- and heavy-chain coding sequences. Cold Spring Harb Protoc 2011(2011).
24. Choi, J. R., et al. BLI-Based Functional Assay in Phage Display Benefits the Development of a PD-L1-Targeting Therapeutic Antibody Viruses 12(2020).
25. Lee, Y., Kim, H. & Chung, J. An antibody reactive to the Gly63-Lys68 epitope of NT-proBNP exhibits O-glycosylation-independent binding. Exp Mol Med 46, e114 (2014).
26. Barbas, C. F., Burton, D., Scott, J. & Silverman, G. Phage display. A Laboratory Manual (2001).

What is claimed is:

1. A composition for predicting or diagnosing Alzheimer's disease, comprising a detecting agent of a B cell receptor (BCR) clonotype specific to an Alzheimer's disease patient in a sample of a subject,
wherein the BCR clonotype comprises the V gene; and the J gene; and an amino acid sequence of complementary determining region 3 (CDR3) of the heavy chain of the BCR.

2. The composition according to claim 1,
wherein when the BCR clonotype detected from the sample of the subject is below i) to iii), the subject is predicted or diagnosed as having Alzheimer's disease:
i) the V gene is at least one selected from the group consisting of IGHV (Immunoglobulin Heavy Variable) 1-18, IGHV1-2, IGHV1-24, IGHV1-3, IGHV1-46, IGHV1-58, IGHV1-69, IGHV1-8, IGHV2-26, IGHV2-5, IGHV2-70, IGHV3-11, IGHV3-13, IGHV3-15, IGHV3-20, IGHV3-21, IGHV3-23, IGHV3-30, IGHV3-30-3, IGHV3-33, IGHV3-43, IGHV3-43D, IGHV3-48, IGHV3-49, IGHV3-53, IGHV3-64, IGHV3-64D, IGHV3-66, IGHV3-7, IGHV3-72, IGHV3-73, IGHV3-74, IGHV3-9, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, and IGHV7-4-1,
ii) the J gene in the BCR clonotype detected from a sample of the subject is at least one selected from the group consisting of IGHJ (Immunoglobulin Heavy Joining) 1, IGHJ2, IGHJ3, IGHJ4, IGHJ5, and IGHJ6, and
iii) the amino acid sequence of CDR3 in the BCR clonotype detected from a sample of the subject is 80 to 100% homologous to at least one selected from the group consisting of SEQ ID NOS: 72 to 4047, and 32396.

3. The composition according to claim 1,
wherein when the BCR clonotype detected from the sample of the subject is at least one of BCR clonotypes of the V gene; the J gene; and the amino acid sequence of CDR3, the subject is predicted or diagnosed as having Alzheimer's disease:

4. The composition according to claim 1,
wherein the detecting agent of a BCR clonotype is a detecting agent of the entire amino acid sequence of the BCR clonotype, comprising an amino acid sequence encoded from the V gene of the heavy chain of the BCR; an amino acid sequence encoded from the J gene of the heavy chain of the BCR; and an amino acid sequence of CDR3 of the heavy chain of the BCR, and
wherein the composition is a composition that when the entire amino acid sequence of the BCR clonotype detected from the sample of the subject is at least one of SEQ ID NOS: 18222 to 32395, the subject is predicted or diagnosed as having Alzheimer's disease.

5. The composition according to claim 1,
wherein the detecting agent of a BCR clonotype is a detecting agent of the entire nucleic acid sequence of the BCR clonotype, comprising a nucleic acid sequence of the V gene of the heavy chain of the BCR; a nucleic acid sequence of the J gene of the heavy chain of the BCR; and a nucleic acid sequence encoding CDR3 of the heavy chain of the BCR, and
wherein the composition is a composition that when the entire nucleic acid sequence of the BCR clonotype detected from the sample of the subject is at least one of SEQ ID NOS: 4048 to 18221, the subject is predicted or diagnosed as having Alzheimer's disease.

6. The composition according to claim 2,
wherein when the amino acid sequence of CDR3 in the BCR clonotype detected from the sample of the subject is 80 to 100% homologous to at least one selected from the group consisting of ARDLPATGAFDI (SEQ ID NO: 214), VRLAEYFQN (SEQ ID NO: 875), TRDRRGWD (SEQ ID NO: 1211), ALGRGMDV (SEQ ID NO: 1048), VLRGSAFDI (SEQ ID NO: 798), ARSGGLDP (SEQ ID NO: 668), AGGRSFDY (SEQ ID NO: 613), AGNLMDV (SEQ ID NO: 2257), ARRGEY (SEQ ID NO: 2070), ARDMFRGIPDYLDY (SEQ ID NO: 1988), AKSLGSGTYSFDY (SEQ ID NO: 1326), ARNAGLDY (SEQ ID NO: 1128), ARDDGYRSIDY (SEQ ID NO: 990), VRGGGSGWPFES (SEQ ID NO: 3709), SRGRDGIG (SEQ ID NO: 2815), ARSGGLDV (SEQ ID NO: 2415), ARSRSGSYYYGMDV (SEQ ID NO: 1198), AGDWNDDDIFDY (SEQ ID NO: 32396), ARKGEQLWHY (SEQ ID NO: 965), and ARDPIAVPGLFDY (SEQ ID NO: 347) the subject is predicted or diagnosed as having Alzheimer's disease.

7. The composition according to claim 1,
wherein the detecting agent comprises a detecting agent of a protein specific to the BCR clonotype or a detecting agent of a nucleic acid encoding the protein.

8. The composition according to claim 7,
wherein the detecting agent of a protein comprises an antibody or aptamer specific to the BCR clonotype.

9. The composition according to claim 7,
wherein the detecting agent of a nucleic acid comprises at least one selected from the group consisting of antisense oligonucleotide, primer pair, probe, and polynucleotide that specifically binds to a mRNA or DNA encoding the BCR clonotype.

10. A system for predicting or diagnosing Alzheimer's disease comprising the composition according to claim 1.

11. A method for providing information for predicting or diagnosing Alzheimer's disease comprising:
detecting a B cell receptor (BCR) clonotype specific to an Alzheimer's disease patient in a sample of a subject,
wherein the BCR clonotype comprises the V gene; and the J gene; and an amino acid sequence of complementary determining region 3 (CDR3) of the heavy chain of the BCR.

12. The method for providing information according to claim 11, further comprising predicting or diagnosing the subject as having Alzheimer's disease when the BCR clonotype detected from the sample of the subject is below i) to iii):
i) the V gene is at least one selected from the group consisting of IGHV (Immunoglobulin Heavy Variable) 1-18, IGHV1-2, IGHV1-24, IGHV1-3, IGHV1-46, IGHV1-58, IGHV1-69, IGHV1-8, IGHV2-26, IGHV2-5, IGHV2-70, IGHV3-11, IGHV3-13, IGHV3-15, IGHV3-20, IGHV3-21, IGHV3-23, IGHV3-30, IGHV3-30-3, IGHV3-33, IGHV3-43, IGHV3-43D, IGHV3-48, IGHV3-49, IGHV3-53, IGHV3-64, IGHV3-64D, IGHV3-66, IGHV3-7, IGHV3-72, IGHV3-73, IGHV3-74, IGHV3-9, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, and IGHV7-4-1,
ii) the J gene in the BCR clonotype detected from a sample of the subject is at least one selected from the group consisting of IGHJ (Immunoglobulin Heavy Joining) 1, IGHJ2, IGHJ3, IGHJ4, IGHJ5, and IGHJ6, and
iii) the amino acid sequence of CDR3 in the BCR clonotype detected from a sample of the subject is 80 to 100% homologous to at least one selected from the group consisting of SEQ ID NOs: 72 to 4047, and 32396.

13. The method for providing information according to claim 11, further comprising predicting or diagnosing the subject as having Alzheimer's disease when the BCR clonotype detected from the sample of the subject is at least one of BCR clonotypes of the V gene; the J gene; and the amino acid sequence of CDR3.

14. The method for providing information according to claim 11, further comprising predicting or diagnosing the subject as having Alzheimer's disease when the entire amino acid sequence of the BCR clonotype detected from the sample of the subject which comprises an amino acid sequence of the V gene of the heavy chain of the BCR; an amino acid sequence of the J gene of the heavy chain of the BCR; and an amino acid sequence of CDR3 of the heavy chain of the BCR is at least one of SEQ ID NOS: 18222 to 32395.

15. The method for providing information according to claim 11, further comprising predicting or diagnosing the subject as having Alzheimer's disease when the entire nucleic acid sequence of the BCR clonotype detected from the sample of the subject is at least one of SEQ ID NOS: 4048 to 18221 detected from the sample of the subject which comprises a nucleic acid sequence of the V gene of the heavy chain of the BCR; a nucleic acid sequence of the J gene of the heavy chain of the BCR; and a nucleic acid sequence encoding CDR3 of the heavy chain of the BCR.

16. The method for providing information according to claim 12, further comprising predicting or diagnosing the subject as having Alzheimer's disease when the amino acid sequence of CDR3 in the BCR clonotype detected from the sample of the subject is 80 to 100% homologous to at least one selected from the group consisting of ARDLPATGAFDI (SEQ ID NO: 214), VRLAEYFQN (SEQ ID NO: 875), TRDRRGWD (SEQ ID NO: 1211), ALGRGMDV (SEQ ID NO: 1048), VLRGSAFDI (SEQ ID NO: 798), ARSGGLDP (SEQ ID NO: 668), AGGRSFDY (SEQ ID NO: 613), AGNLMDV (SEQ ID NO: 2257), ARRGEY (SEQ ID NO: 2070), ARDMFRGIPDYLDY (SEQ ID NO: 1988), AKSLGSGTYSFDY (SEQ ID NO: 1326), ARNAGLDY (SEQ ID NO: 1128), ARDDGYRSIDY (SEQ ID NO: 990), VRGGGSGWPFES (SEQ ID NO: 3709), SRGRDGIG (SEQ ID NO: 2815), ARSGGLDV (SEQ ID NO: 2415), ARSRSGSYYYGMDV (SEQ ID NO: 1198), AGDWNDDDIFDY (SEQ ID NO: 32396), ARKGEQLWHY (SEQ ID NO: 965), and ARDPIAVPGLFDY (SEQ ID NO: 347).
